(19) United States
(12) Patent Application Publication
(10) Pub. No.: US 2024/0109878 A1
(43) Pub. Date: Apr. 4, 2024

(54) COMPOUND AND ITS USE IN ORGANIC ELECTRONIC DEVICES

(71) Applicant: HELIATEK GMBH, Dresden (DE)

(72) Inventors: Olga Gerdes, Ulm (DE); Martin Pfeiffer, Dresden (DE); Andre Weiss, Dresden (DE); Ivan Ramirez, Dresden (DE)

(21) Appl. No.: 18/260,056

(22) PCT Filed: Dec. 30, 2021
(86) PCT No.: PCT/EP2021/087875

§ 371 Date: Jun. 30, 2023

(30) Foreign Application Priority Data
Dec. 31, 2020 (DE) 10 2020 135 172.0

(51) Int. Cl. C07D 413/14; C09K 11/06; H10K 85/60

(52) U.S. Cl. CPC C07D 413/14; C09K 11/06; H10K 85/656

(57) ABSTRACT

A compound is provided that can be evaporated largely without residue in a vacuum. Because the compound can be evaporated largely without residue in a vacuum, the compound is thus suitable for vacuum processing for producing solar cells. The compound advantageously demonstrates a blue shift in the absorption spectrum. An organic electronic device including the compound is also provided.

### CROSS REFERENCE TO RELATED APPLICATIONS

This application is a U.S. National Phase application under 35 U.S.C. § 371 of International Application No. PCT/EP2021/087875, filed on Dec. 30, 2021, and claims benefit to German Patent Application No. DE 10 2020 135 172.0, filed on Dec. 31, 2020. The International Application was published in German on Jul. 7, 2022 as WO 2022/144429 A1 under PCT Article 21(2).

### FIELD

The invention relates to a compound of the general formula (I), a use of such a compound in an organic electronic device, and an organic electronic device comprising such a compound.

### BACKGROUND

Electronic devices made of organic materials are known for use as LED's (OLED) and organic photovoltaic elements (OPV), i.e., organic solar cells. The organic materials used fulfill different functions in these organic electronic devices—in particular, charge transport, light emission, or light absorption. Organic materials in opto-electronic devices can be polymers or small molecules and can be processed in solution or emulsion by means of wet-chemical processes such as coating or printing, or in a vacuum by sublimation to form thin layers. Organic electronic devices can, for example, be displays, data memories, or transistors, but also organic optoelectronic devices—in particular, solar cells or photodetectors. Solar cells or photodetectors have a photoactive layer in which bound electron-hole pairs (excitons) are produced as charge carriers when electromagnetic radiation is applied. The excitons pass by diffusion to an interface at which electrons and holes are separated from one another. The material which absorbs the electrons is referred to as an acceptor, and the material which absorbs the holes is referred to as a donor. Further organic electronic devices are light-emitting devices which emit light when current is passed through them. Organic electronic devices comprise at least two electrodes, wherein one electrode is applied to a substrate, and the other functions as a counter-electrode. Between the electrodes there is at least one photoactive layer—preferably an organic photoactive layer. Further layers, e.g., transport layers, can be arranged between the electrodes.

Organic semiconductive materials are still being sought which, when used in organic electronic devices, lead to an improvement in the properties of the devices. The absorption spectrum of known absorbers does not completely cover the blue spectral range. For increasing the absorption range and thus for increasing the efficiency of solar cells, absorbers are therefore required which absorb strongly in the spectral range between 400 nm and 600 nm, and in particular between 450 and 550 nm, and have a voltage in the range of 1 V. This is particularly advantageous for use in tandem and triplet cells.

### SUMMARY

In an embodiment, the present disclosure provides a compound of the general formula I

A1-(T1)ₐ-(T2)_{b}-(Z)_{c}-(T3)_{d}-(T4)ₑ-A2 (I),
wherein the parameters a, b, d, and e are each independently 0 or 1. At least one of the parameters a, b, d, and e is equal to 1. The parameter c is equal to 1, 2, or 3. The general group Z is a block of two groups M and N, linked as *-M-N—*or *—N-M-*, wherein * denotes an attachment to groups T1, T2, T3, T4, A1, and A2. The groups M are each independently selected from the following formulas:

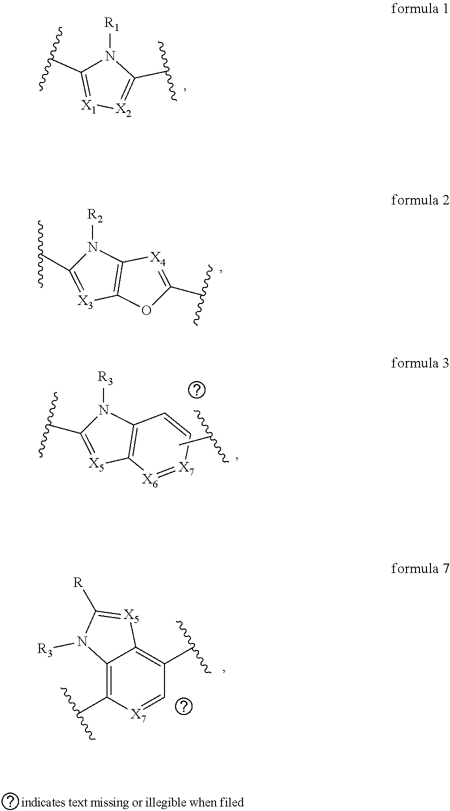

wherein the groups N are each independently selected from the following formulas:

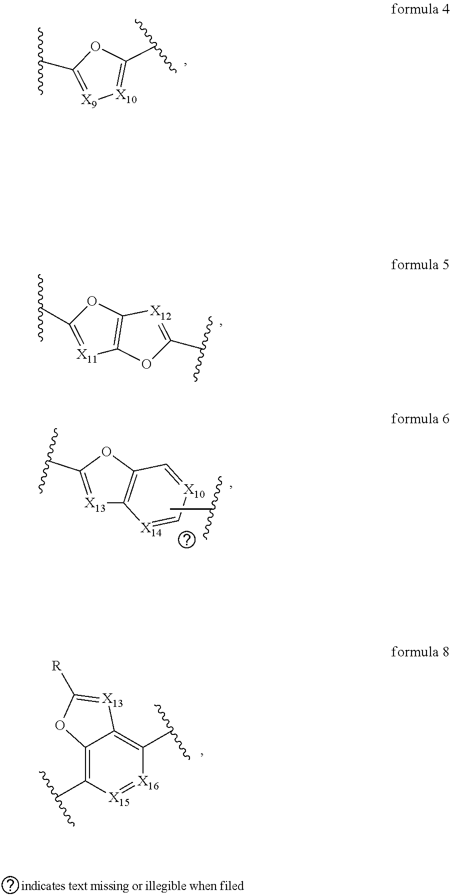

wherein M and N are each linked such that at least one N atom of the group M and an O atom of the group N are each bonded to one another via 2 C atoms. [symbol] denotes the attachment to the groups in the compound of the general formula I, in which X₁-X₁₆ are independently selected from N or C—R, and, in each of the groups of the formulas 3 and 6, a group from the groups X₈/X₇ and X₁₆/X₁₅ denotes the attachment [symbol] to other groups in the compound of the general formula I. R is in each case independently selected from the group consisting of H, halogen, branched or linear, cyclic or open-chain C₁-C₁₀ alkyl, C₂-C₁₀ alkenyl, O-alkyl, S-alkyl, O-alkenyl, S-alkenyl, alkynyl, aryl, and heteroaryl, wherein, in all of the groups from which R is selected, H atoms can be substituted, CN, NR₁₀R₁₁, with R₁₀ and R₁₁ each independently selected from H, branched or linear, and cyclic or open-chain C₁-C₁₀ alkyl, wherein H atoms of the C₁-C₁₀ alkyl can be substituted, and C atoms of the C₁-C₁₀ alkyl can be substituted by heteroatoms. R₁ to R₃ are each independently selected from the group consisting of H, branched or linear, and cyclic or open-chain C₁-C₁₀ alkyl, substituted or unsubstituted C₂-C₁₀ alkenyl, substituted or unsubstituted aryl, substituted or unsubstituted heteroaryl, and CN, wherein H atoms of the C₁-C₁₀ alkyl can be substituted, and C atoms of the C₁-C₁₀ alkyl can be substituted by heteroatoms. A1 and A2 are, independently, electron-withdrawing groups having at least one C═C double bond. The groups T1, T2, T3, and T4 are each independently selected from the following formulas:

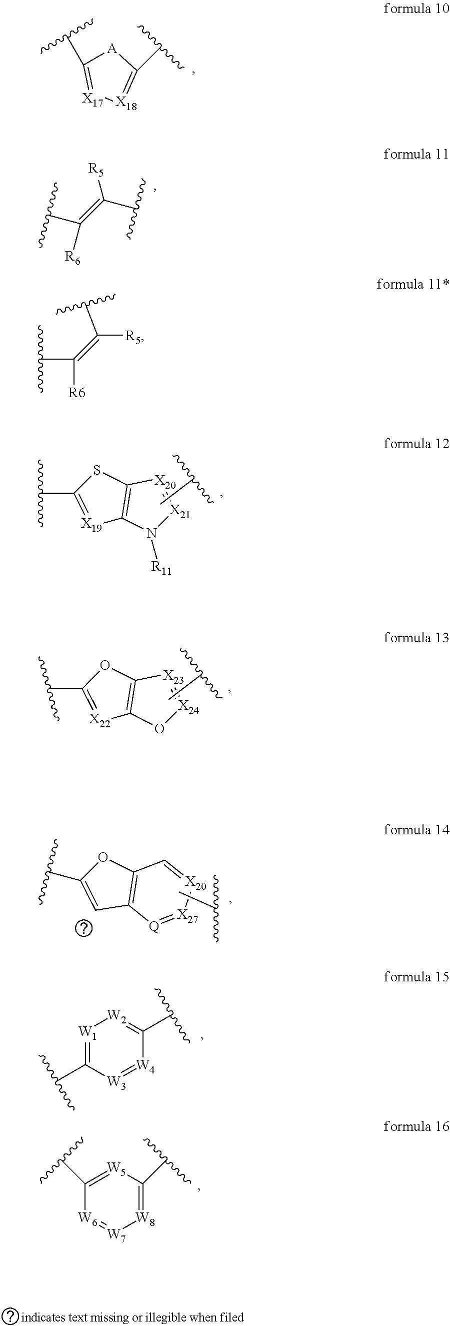

wherein [symbol] denotes the attachment to the other groups in the compound of the general formula I, and at least one of the groups T1 to T4 is the formula 10, in which R₅ and R₆ are each independently selected from the group consisting of H, CN, F, aryl, heteroaryl, C₂-C₁₀ alkenyl, alkynyl, branched or linear, cyclic or open-chain C₁-C₁₀ alkyl, wherein H atoms of the C₁-C₁₀ alkyl can be substituted, wherein, if the substituent R₁₃ is present in the compound of the formula I, a ring closure between R₅ with R₁₃ or R₆ with R₁₃ is possible, and, between R₅ and R₁₃ or between R₆ and R₁₃, in each case there is located the double bond from formula 11. W₁ to W8 are each independently selected from N, CR. X₁₇ and X₁₈ are independently selected from N and C—R, and at least X₁₇ or X₁₈ is N. X₁₉ to X₂₇ are independently selected from N and C—R, and, in each of the groups of the formulas 12, 13, and 14, a group from the groups X₂₀/X₂₁, X₂₃/X₂₄, and X₂₆/X₂₇ denotes the attachment [symbol] to other groups in the compound of the general formula I, in which A is selected from the group consisting of S, O, NR₉, and Se, and Q is selected from the group consisting of S, O, NR₉, and Se. For the groups A and Q, the substituent R₉ is in each case independently selected from H, CN, branched or linear, cyclic or open-chain C₁-C₁₀ alkyl, C₂-C₁₀ alkenyl, O-alkyl, S-alkyl, O-alkenyl, S-alkenyl, alkynyl, substituted or unsubstituted aryl, and substituted or unsubstituted heteroaryl, wherein the H atoms of the C₁-C₁₀ alkyl may be substituted.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 shows a schematic illustration of an exemplary embodiment of an organic electronic device in cross-section;

FIG. 2 shows a current-voltage curve of an organic electronic device having the compound (12);

FIG. 3 shows a current-voltage curve of an organic electronic device having the compound (22); and

FIG. 4 shows a current-voltage curve of an organic electronic device having the compound (35).

### DETAILED DESCRIPTION

In an embodiment, a compound is provided of the general formula I

A1-(T1)ₐ-(T2)_{b}-(Z)_{c}-(T3)_{d}-(T4)ₑ-A2 (I)
with the parameters a, b, d, e each independently 0 or 1, with the proviso that at least one of the parameters a, b, d, e=1,
with the parameter c=1, 2, or 3,
wherein the general group Z is a block of two groups M and N, linked as *-M-N—* or *—N-M-*,
wherein * denotes the attachment to the groups T1, T2, T3, T4, A1, and A2,
wherein the groups M are each independently selected from:

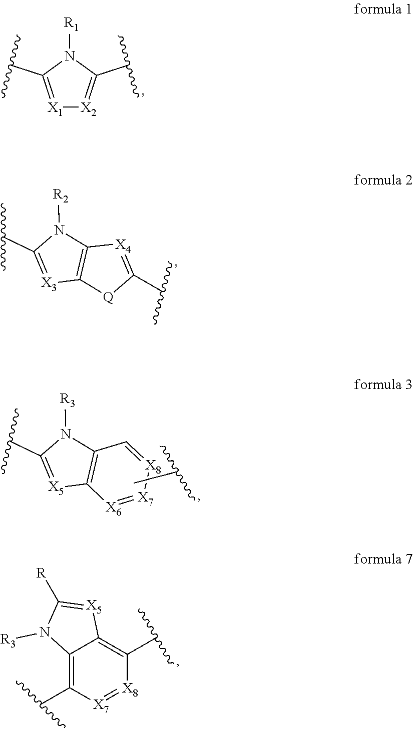

wherein the groups N are each independently selected from:

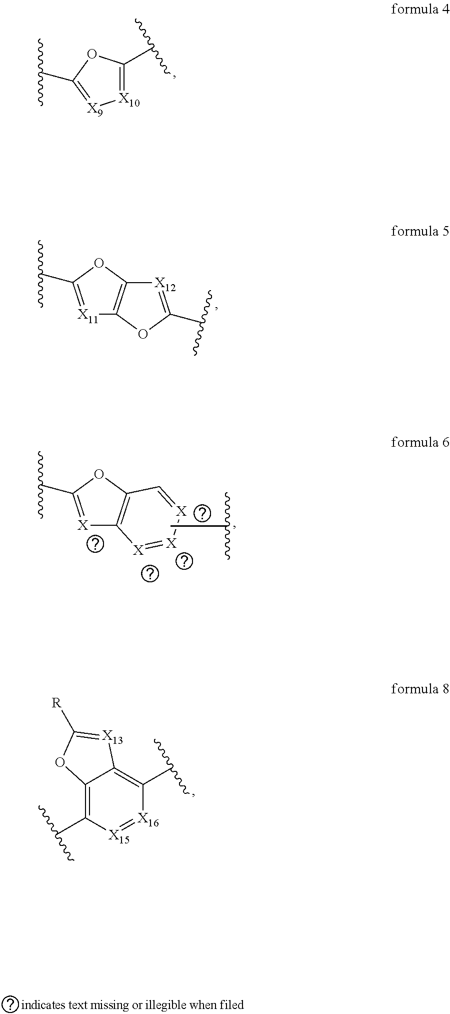

wherein M and N are each linked such that at least one N atom of the group M and an O atom of the group N are each bonded to one another via 2 C atoms, and [symbol] denotes the attachment to the other groups in the compound of the general formula I,
with X₁-X₁₆ independently selected from N or C—R, with the proviso that, in each of the groups of the formulas 3 and 6, a group from the groups X₈/X₇ and X₁₆/X₁₅ denotes the attachment [symbol] to the other groups in the compound of the general formula I,
with R in each case independently selected from the group consisting of H, halogen, branched or linear, cyclic or open-chain C₁-C₁₀ alkyl, wherein H atoms of the C₁-C₁₀ alkyl can be substituted, and C atoms of the C₁-C₁₀ alkyl can be substituted by heteroatoms, C₂-C₁₀ alkenyl, O-alkyl, S-alkyl, O-alkenyl, S-alkenyl, alkynyl, aryl, heteroaryl, wherein, in all of these groups, H atoms can be substituted, CN, NR₁₀R₁₁, with R₁₀ and Ru each independently selected from H, branched or linear, cyclic or open-chain C₁-C₁₀ alkyl, wherein H atoms of the C₁-C₁₀ alkyl can be substituted, and C atoms of the C₁-C₁₀ alkyl can be substituted by heteroatoms,
with R₁ to R₃ each independently selected from the group consisting of H, branched or linear, cyclic or open-chain C₁-C₁₀ alkyl, wherein H atoms of the C₁-C₁₀ alkyl can be substituted, and C atoms of the C₁-C₁₀ alkyl can be substituted by heteroatoms, substituted or unsubstituted C₂-C₁₀ alkenyl, substituted or unsubstituted aryl, substituted or unsubstituted heteroaryl, CN,
wherein the electron-withdrawing groups A1 and A2 are, independently, electron-withdrawing groups having at least one C═C double bond,
wherein the groups T1, T2, T3, and T4 are each independently selected from:

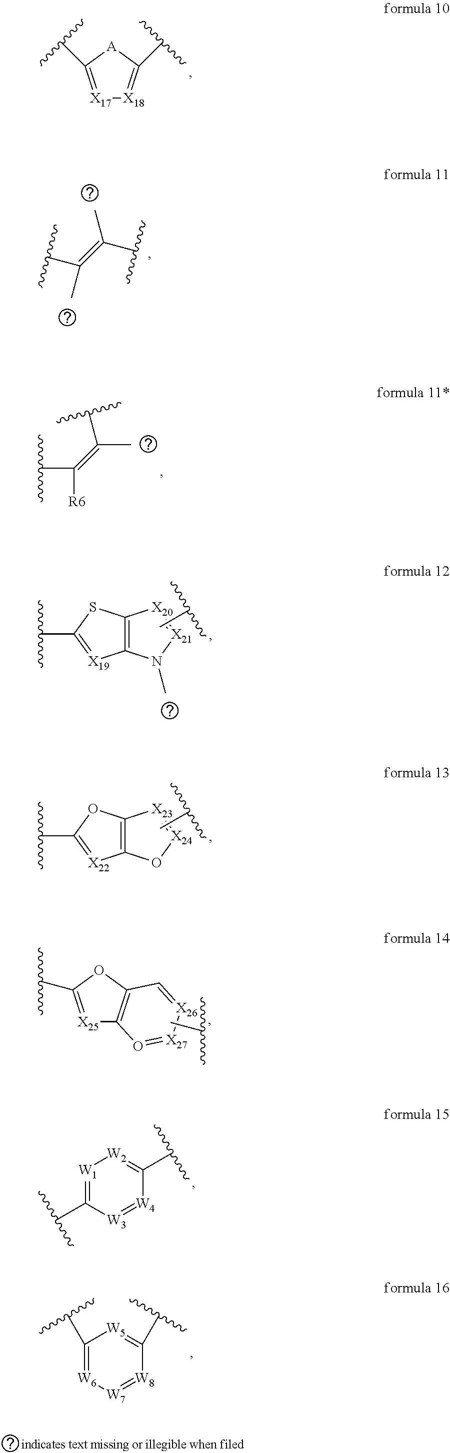

wherein [symbol] denotes the attachment to the other groups in the compound of the general formula I, with the proviso that at least one of the groups T1 to T4 is the formula 10,
with R₅ and R₆ each independently selected from the group consisting of H, CN, F, aryl, heteroaryl, C₂-C₁₀ alkenyl, alkynyl, branched or linear, cyclic or open-chain C₁-C₁₀ alkyl,
wherein H atoms of the C₁-C₁₀ alkyl can be substituted, wherein, if the substituent R₁₃ is present in the compound of the formula I, a ring closure between R₅ with R₁₃ or R₆ with R₁₃ is possible, with the proviso that, between R₅ and R₁₃ or between R₆ and R₁₃, in each case there is located the double bond from formula 11,
with W₁ to W₈ each independently selected from N, CR, wherein R is defined as described above,
with X₁₇ and X₁₈ independently selected from N and C—R, wherein R is defined as described above, with the proviso that at least X₁₇ or X₁₈ is N,
with X₁₉-X₂₇ independently selected from N and C—R, wherein R is defined as described above, and with the proviso that, in each of the groups of the formulas 12, 13, and 14, a group from the
groups X₂₀/X₂₁, X₂₃/X₂₄, and X₂₆/X₂₇ denotes the attachment [symbol] to the other groups in the compound of the general formula I,
with A selected from the group consisting of S, O, NR₉, and Se,
with Q selected from the group consisting of S, O, NR₉, and Se,
wherein, for the groups A and Q, the substituent R₉ is in each case independently selected from H, CN, branched or linear, cyclic or open-chain C₁-C₁₀ alkyl, wherein the H atoms of the C₁-C₁₀ alkyl may be substituted, C₂-C₁₀ alkenyl, O-alkyl, S-alkyl, O-alkenyl, S-alkenyl, alkynyl, substituted or unsubstituted aryl, substituted or unsubstituted heteroaryl.

The conjugated π-electron system of the donor region of the compounds of the formula I according to embodiments of the invention can thereby be expanded beyond the donor block Z in that at least one further donor block T1, T2, T3, or T4 is incorporated, and accordingly the parameters a, b, d or e associated with these donor blocks are successively set to 1 in the formula I.

It is possible for several M-N or N-M block pairs also to follow one another in the central group Z if the parameter c is >1, e.g., *-M-N-M-N-M-N—*, *-M-N—N-M-M-N—*, or *—N-M-N-M-N-M-N-M-*. Due to this structural feature (*-M-N—*)_{c} or (*—N-M-*)_{c}, the present compounds have a high optical density, preferably in the visible spectral range, and in particular a high integral over the optical density in the absorption spectrum compared to compounds not according to embodiments of the invention which do not have the structural element described above. Here, an “integral” is understood to mean the area content below a curve in the absorption spectrum, which is an important feature for the suitability of the material as organic photosensitive material.

In a preferred embodiment of the invention, R is selected from the group consisting of H, halogen, and a branched or linear, cyclic or open-chain C₁-C₁₀ alkyl, wherein preferably H atoms of the C₁-C₁₀ alkyl are partially or completely substituted by F.

In a preferred embodiment of the invention, at least one H atom of formula 9 is substituted, preferably substituted by a halogen, preferably F, an alkyl group, wherein the alkyl group is unsubstituted or at least one H atom is substituted by a halogen, preferably F, or an O-alkyl group.

In a preferred embodiment of the invention, at least one T1 to T4 is a substituted or unsubstituted thiazole, and at least one T1 to T4 is a substituted or unsubstituted furan or thiophene.

In a preferred embodiment of the invention, the group M is pyrrole and/or the group N is furan—in each case substituted or unsubstituted.

The chemical compounds according to embodiments of the invention of the general formula I have advantages compared to the prior art. Advantageously, improved absorbers for organic electronic devices, and in particular solar cells, can be provided. Advantageously, absorber materials are provided, for the red and near-infrared spectral range, with a high absorption strength. The compounds according to embodiments of the invention are advantageously characterized by a broad absorption range shifted into the blue range. The compounds according to embodiments of the invention advantageously demonstrate surprisingly good absorption behavior in a comparatively wide range of visible light from 400 nm to 800 nm, and preferably from 400 to 700 nm—in particular, a high absorption in the short-wavelength spectral range of 400 nm to 600 nm. Advantageously, the open-circuit voltage Uoc is increased. Advantageously, the efficiency of solar cells can be increased.

Advantageously, the compounds according to embodiments of the invention can be evaporated largely without residue in a vacuum, and are thus suitable for vacuum processing for producing solar cells. The compounds according to embodiments of the invention advantageously demonstrate a blue shift in the absorption spectrum—in particular, compounds according to embodiments of the invention with at least one thiazole of the groups T1 to T4, compared to furan or thiophene at this position. Advantageously, the open-circuit voltage Uoc is in the region of 1 V or higher. This is particularly advantageous in the case of multiple cells, and preferably tandem or triplet cells, or mixed layers, since the smallest voltage of the layer is limiting for the total voltage of the cell. Advantageously, compounds according to embodiments of the invention, in comparison to corresponding compounds without at least one T1, T2, T3, and T4 with formula 10 with at least one X₁₇ or X₁₈ an N, demonstrate an increased open-circuit voltage Uoc with simultaneously strong, sufficiently long-wavelength absorption (onset >700 nm) and a high fill factor—preferably >60%, and particularly preferably >64%—for bulk heterojunctions with a fullerene—preferably C60.

The compounds according to embodiments of the invention are in particular so-called “small molecules,” wherein are thereby meant non-polymeric oligomeric organic molecules with a molar mass of between 100 and 2000 g/mol, which, in particular, can also be monodisperse.

In a preferred embodiment of the invention, T1, T2, T3, or T4, and in particular T1 or T4, is a thiazole, an oxazole, a thiadiazole, or an oxadiazole.

According to an embodiment of the invention, it is provided that the electron-withdrawing groups A1 and A2 be selected independently from:

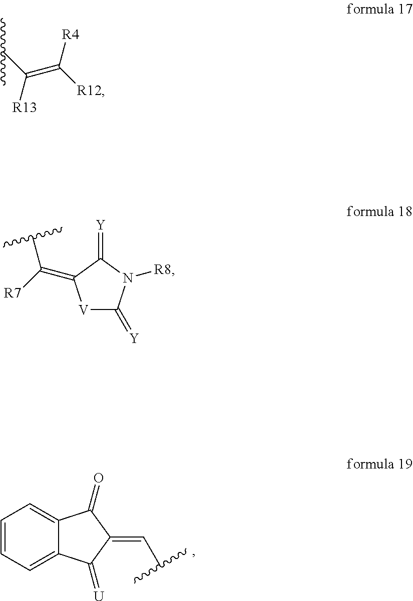

and [symbol] denotes the attachment to the groups T1 to T4 and Z in the compound of the general formula I,
with R₄ and R₁₂ each independently selected from H, CN, and COOR, with the proviso that R₄ and R₁₂ are not both H,
wherein R is selected from the same group of compounds as defined in R₁ to R₃,
with R₁₃ in each case independently selected from the group consisting of H, CN, F, aryl, heteroaryl, C₂-C₁₀ alkenyl, alkynyl, branched or linear, cyclic or open-chain C₁-C₁₀ alkyl,
wherein H atoms of the C₁-C₁₀ alkyl can be substituted, wherein, if the substituent R₅ or R₆ is present in the compound of the formula I, a ring closure between R₅ with R₁₃ or R₆ with R₁₃ is possible, with the proviso that, between R₅ and R₁₃ or between R₆ and R₁₃, in each case there is located the double bond from formula 11,
with V═O, S; with Y═O, S, C(CN)₂; with U═O, S, C(CN)₂,
with R₇ and R₈ each independently selected from the group consisting of H, CN, F, aryl, heteroaryl, C₂-C₁₀ alkenyl, alkynyl, branched or linear, cyclic or open-chain C₁-C₁₀ alkyl, wherein H atoms of the C₁-C₁₀ alkyl may be substituted, wherein, for each of the groups A1 and A2, in each case for each C═C double bond in each case independently, both the E-isomer and the Z-isomer can be present.

For each C═C double bond of the formulas 7, 8, and 9, therefore, both the E-isomer (“E”=opposite, i.e., trans-configuration) and also the Z-isomer (“Z”=together, i.e., cis-configuration) are present, wherein these isomers are formed by an imaginary rotation through 180° about the C═C double-bond axis. This will be explained hereafter on the basis of the example of the residue of formula 18:

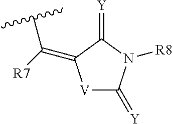

the two isomers, which can be present separately from one another, can be converted into one another by an imaginary rotation about the C═C double bond (indicated by the arrow at the double bond), so that the following two isomers result for the group of formula 18:

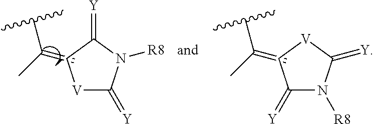

In a preferred embodiment of the invention, A1 is equal to A2.

In the compounds of the general formula I according to embodiments of the invention, the aryl groups and the heteroaryl groups can preferably be C₅-C₁₀ aryl and C₅-C₁₀ heteroaryl groups. Substituents are understood to be all atoms and atom groups apart from H. Potential substituents are, in particular, halogen, and preferably fluorine, but also C₁-C₅ alkyl groups, which may in turn be substituted. The O-alkyl, S-alkyl, O-alkenyl, S-alkenyl, alkynyl groups may each be C₁-C₁₀ groups, and preferably C₁-C₅ groups.

The cyclic or open-chain C₁-C₁₀ alkyl groups of the compounds of the formula I according to embodiments of the invention can be linear or else branched, and are preferably C₁-C₅ alkyl groups. Non-adjacent and non-terminal C atoms of these alkyl groups can be replaced by heteroatoms.

“Heteroatoms” in the sense of the present compounds of formula I are understood in particular to mean O, S, Se, or NR, wherein the substituent R is defined as the substituents R₁ to R₃ which have already been described above.

The bonding points in the individual groups, which are denoted by [symbol] , characterize the attachment points of the respective groups to the other groups of the compounds of formula I, i.e., for example, in the electron-withdrawing group A1 in the compound of formula I, the attachment either to the donor groups T1 (at a=1), or T2 (at a=0 and b=1), or to the donor group Z when the parameters a and b are both 0.

According to an embodiment of the invention, it is provided that the electron-withdrawing groups A1 or A2 be the following group

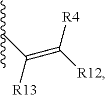

wherein preferably R4 and R12 are CN. Such electron-withdrawing groups A1 and A2 lead to oligomeric compounds of formula I, which can be applied to substrates particularly well by vapor deposition. Particularly preferably, R4 and R12 are CN, so that the particularly strongly electron-withdrawing group dicyanovinylene results. Furthermore, the substituent R13 can preferably be H.

According to an embodiment of the invention, it is provided that one of the parameters a, b, d, e=0, preferably two of the parameters a, b, d, e=0, and particularly preferably three of the parameters a, b, d, e=0, and/or at least one of the parameters a, b=1 and/or one of the parameters d, e=1, preferably one of the parameters a, b=0 and one of the parameters d, e=0, and particularly preferably a and b=1 and d and e=1.

According to an embodiment of the invention, it is provided that, in the general formula I, the parameter c=1 is

A1-(T1)ₐ-(T2)_{b}-Z-(T3)_{d}-(T4)ₑ-A2 (II).

The inventors have found that one donor block Z is sufficient to achieve an increased optical density with respect to structurally different compounds.

According to an embodiment of the invention, it is provided that Z=*-M-N—* or *—N-M-*, and M is a group of

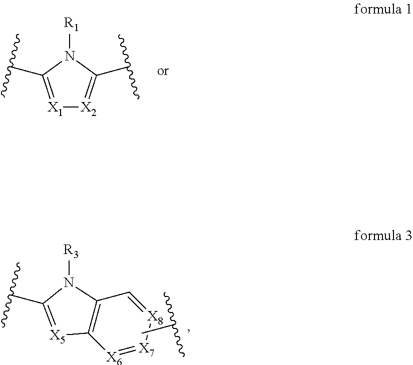

wherein preferably X₁ and X₂ are independently selected from C—R with R in each case independently selected from the group consisting of H, halogen, branched or linear, cyclic or open-chain C₁-C₁₀ alkyl.

According to an embodiment of the invention it is provided that, in the general formula I, the parameter c=1 is

A1-(T1)ₐ-(T2)_{b}-Z-(T3)_{d}-(T4)ₑ-A2 (II),
wherein preferably T1 or T2, and T3 or T4 are, independently, formula 10. These simple pyrrole structural units for the donor block M, which do not have any further condensed aromatic π-electron system, lead already to a noticeable increase in the absorption of radiation for the compounds according to embodiments of the invention if they also have the donor group N.

In the context of embodiments of the present invention, the terms, “substituted” and “substituent,” are to be interpreted such that one or more H atoms are replaced with any other atom group or another atom. In this sense, “substituents” can in particular be a halogen or a pseudohalogen—preferably fluorine or CN—and an aryl group—preferably phenyl—or an alkyl group—preferably a C₁-C₆ alkyl group.

The general groups and substituents in the donor block M of the general formula I can be defined as follows:
X₁ and X₂ independently selected from C—R with R in each case independently selected from the group consisting of H, halogen, branched or linear, cyclic or open-chain C₁-C₁₀ alkyl.

These simple furan structural units for the donor block N, which do not have any further condensed aromatic π-electron system, lead already to a noticeable increase in the absorption of radiation for the compounds according to embodiments of the invention if they also have the donor group M. However, it is also possible to use condensed donor blocks which contain furan, such as benzofurans or other compounds falling under the general formulas 5 or 6. Here, the general groups X₉ and X₁₀ in formula 4 can preferably be, independently, C—R with R in each case independently selected from the group consisting of H, halogen, branched or linear, cyclic or open-chain C₁-C₁₀ alkyl.

According to an embodiment of the invention, it is provided that X₁₇ or X₁₈═C—R, wherein R is independently selected from a group consisting of H, halogen, branched or linear, cyclic or open-chain C₁-C₁₀ alkyl, R₅ and R₆ are each independently selected from H, CN, F, aryl, heteroaryl, C₂-C₁₀ alkenyl, alkynyl, branched or linear, cyclic or open-chain C₁-C₁₀ alkyl, wherein H atoms of the C₁-C₁₀ alkyl can be substituted.

If the substituent R₅ and R₆ is present in the compound, a ring closure between R₅ with R₁₃ as well as between R₆ with R₁₃ is possible, with the proviso that, between R₅ and R₁₃ or between R₆ and R₁₃, there is located the double bond from formula 11 or formula 11*.

According to an embodiment of the invention, it is provided that X₁₇ and X₁₈ be N, and/or X₁₉ to X₂₇ be independently selected from C—R, wherein R is defined as described above, and with the proviso that, in each of the groups of the formulas 12, 13, and 14, a group from the groups X₂₀/X₂₁, X₂₃/X₂₄, and X₂₆/X₂₇ denotes the attachment [symbol] to the other groups in the compound of the general formula I.

According to an embodiment of the invention, the group N is the following general group of formula 4

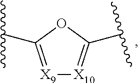

wherein preferably X₉ and X₁₀ are independently selected from C—R with R in each case independently selected from the group consisting of H, halogen, branched or linear, cyclic or open-chain C₁-C₁₀ alkyl.

According to an embodiment of the invention, it is provided that M be the general group of formula 3 and/or N be the general group of formula 6, wherein preferably, in formula 10, A=S or O.

According to an embodiment of the invention, it is provided that b=1 and T2 be the group of formula 10

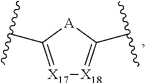

wherein preferably, in formula 10, A=S or O, and/or wherein d=1 and T3 is the group of formula 10

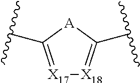

or formula 11

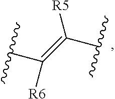

wherein preferably, in formula 10, A=S or O.

According to an embodiment of the invention, it is provided that e=1 and T4 be the group of formula 10

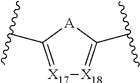

or formula 11

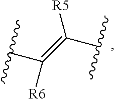

wherein preferably, in formula 10, A=O or S, and/or wherein a=1 and T1 is the group of formula 10

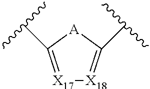

or formula 11

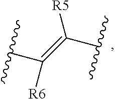

wherein preferably, in formula 10, A=S or O.

According to an embodiment of the invention it is provided that the compound be selected from the group consisting of:

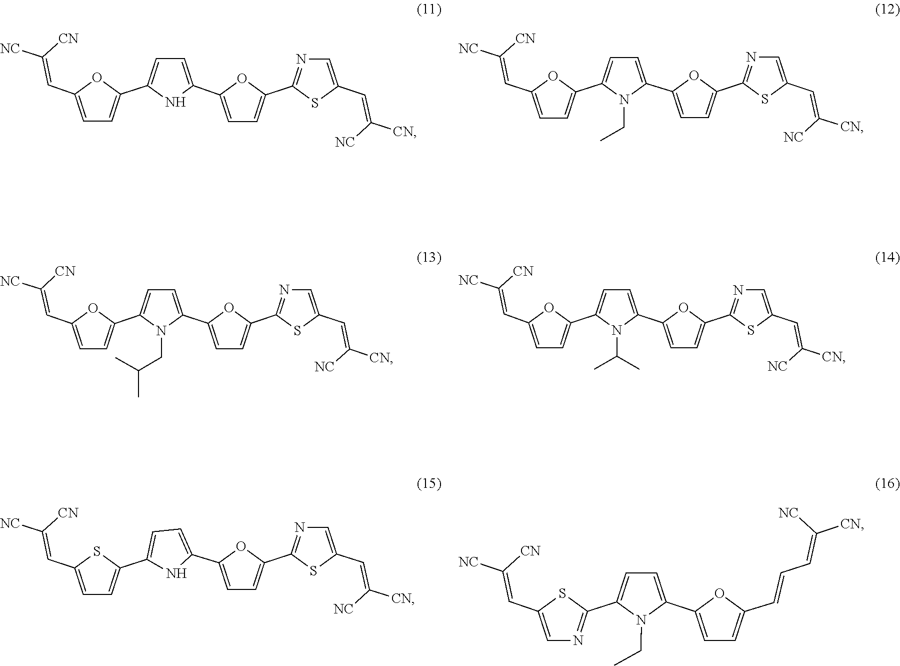 [SMILES: N#CC(C#N)=Cc1ccc(-c2ccc(-c3ccc(-c4ncc(C=C(C#N)C#N)s4)o3)[nH]2)o1 ; CCn1c(-c2ccc(C=C(C#N)C#N)o2)ccc1-c1ccc(-c2ncc(C=C(C#N)C#N)s2)o1 ; CC(C)Cn1c(-c2ccc(C=C(C#N)C#N)o2)ccc1-c1ccc(-c2ncc(C=C(C#N)C#N)s2)o1 ; CC(C)n1c(-c2ccc(C=C(C#N)C#N)o2)ccc1-c1ccc(-c2ncc(C=C(C#N)C#N)s2)o1 ; N#CC(C#N)=Cc1ccc(-c2ccc(-c3ccc(-c4ncc(C=C(C#N)C#N)s4)o3)[nH]2)s1 ; CCn1c(-c2ccc(/C=C/C=C(C#N)C#N)o2)ccc1-c1ncc(C=C(C#N)C#N)s1]

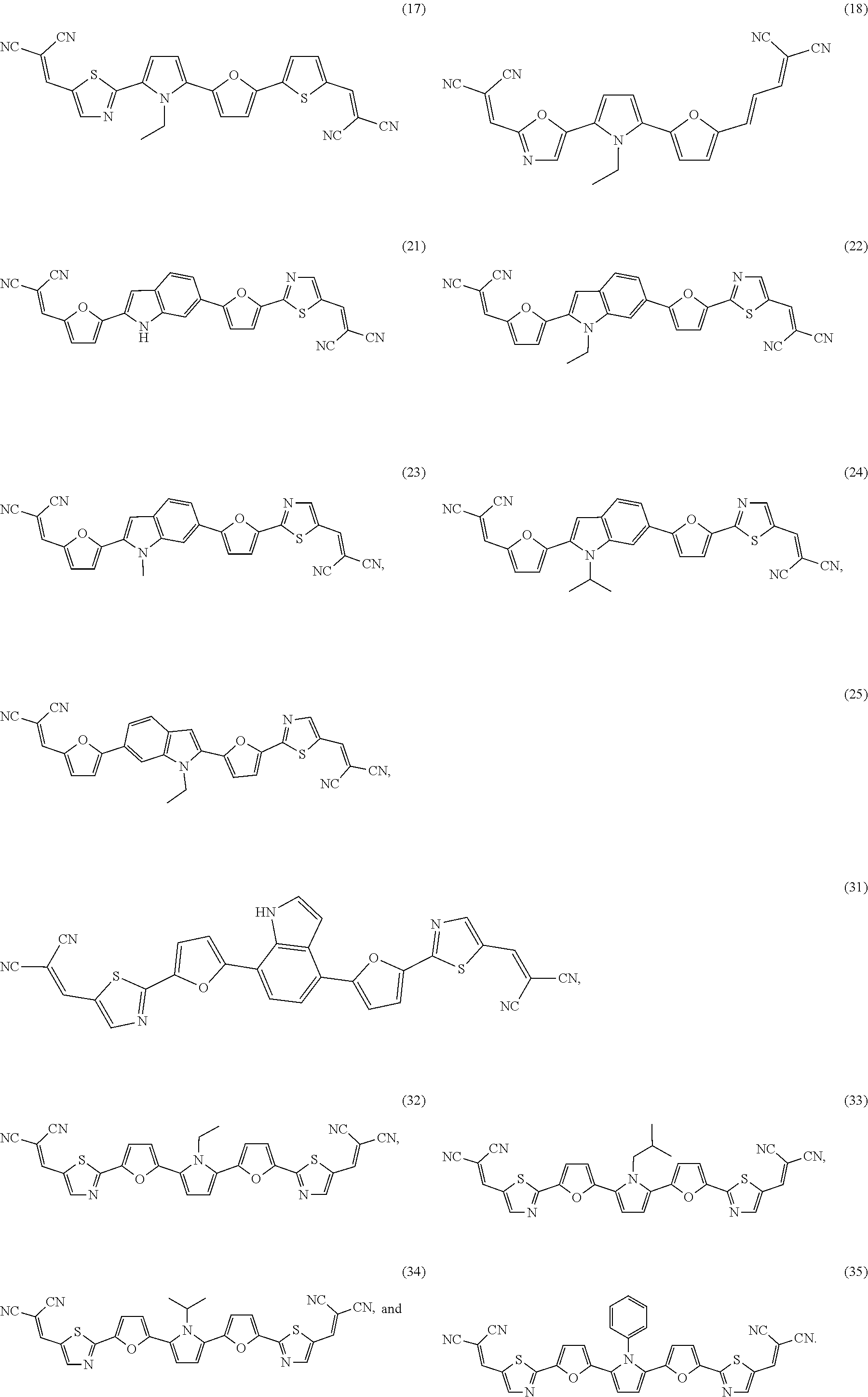

According to an embodiment of the invention, it is provided that c=1, b=1, and/or e=1.

Owing to the particularly strong absorption of the compounds according to embodiments of the invention, excitons are formed particularly well in layers which comprise these compounds, which, in the case of organic photoactive devices comprising these compounds, leads to higher fill factors FF, improved open-circuit voltage V_{oc}, and improved short-circuit current density J_{sc}. In other organic electronic apparatuses, better electronic values are also to be expected due to the increased charge carrier transport properties of the compounds according to embodiments of the invention.

In an embodiment, the present invention provides an organic electronic device comprising at least one compound according to embodiments of the invention, and in particular according to one of the exemplary embodiments described above. In particular, the advantages that have already been explained in conjunction with the compound of the general formula (I) are provided for the organic electronic device.

According to an embodiment of the invention, it is provided that the organic electronic device has a layer system, wherein the layer system comprises an electrode, a counter-electrode, and at least one photoactive layer, wherein the at least one photoactive layer is arranged between the electrode and the counter-electrode.

An organic electronic device is understood to mean in particular an electronic device which has organic conductive or semiconductive materials, and in particular transistors, organic light-emitting devices, organic photoactive apparatuses, in which excitons (electron-hole pairs) can be formed in a photoactive layer by means of irradiation—preferably photodetectors and solar cells.

In a preferred embodiment of the invention, the at least one photoactive layer has at least one compound according to embodiments of the invention.

According to an embodiment of the invention, it is provided that the organic electronic device be an organic solar cell, an OFET, an OLED, or an organic photodetector.

In a preferred embodiment of the invention, the organic electronic device is formed as a tandem or multiple cell, wherein at least one further absorber material which absorbs in a different spectral range of light is provided. The tandem cell refers here in particular to a solar cell which consists of a vertical layer system of two cells interconnected in series. The multiple solar cell refers in particular to a solar cell which consists of a vertical layer system of several cells interconnected in series.

In a preferred embodiment of the invention, the compound of general formula (I) according to embodiments of the invention is an absorber material in a photoactive layer of an organic electronic device. In a preferred embodiment of the invention, the compound according to embodiments of the invention is a donor in a donor-acceptor heterojunction—preferably used with an acceptor selected from the group of fullerenes (C60, C70) or fullerene derivatives, subphthalocyanines, rylenes, fluorenes, carbazoles, benzothiadiazoles, diketopyrrolopyrroles, and vinazenes.

The photoactive layer can perform here a function that is important for the electronic function of the organic device, e.g., a charge carrier-transporting function, such as the transport of holes (p-conducting) or the transport of electrons (n-conducting). Furthermore, the photoactive layer can also comprise a light-emitting layer which, when a voltage is applied to the electrode and counter-electrode, emits radiation, e.g., light, by recombination of the holes (positive charges) and electrodes (negative charge). The organic functional layer can also be a photoactive layer in which excitons (electron-hole pairs) are formed during irradiation with radiation, e.g., light, or else UV radiation or IR radiation. In the case of organic photoactive layers, so-called planar heterojunctions can be formed in particular, in which case a planar p-conducting layer is present adjacent to a planar n-conducting layer, and the excitons formed by irradiation either in the p-conducting or n-conducting layer can be separated into holes and electrons at the interface between the two layers. Furthermore, the photoactive layer can also comprise a so-called bulk heterojunction, in which p-conducting and n-conducting materials transition into one another in the form of an inter-penetrating network, wherein there too the separation of the excitons formed by irradiation occurs at the interfaces between p-conducting and n-conducting materials.

Excitons are electrically neutral excitation states, the electron-hole pairs, that are then separated in a further step at a p-n junction into electrons and holes. The separation into free charge carriers, which contribute to the electrical current flow, thus takes place. In this case, the size of the band gap of the semiconductor is limiting, and accordingly only photons which have an energy which is greater than the band gap can be absorbed. The light always generates excitons first, and no free charge carriers; accordingly, the low-recombination diffusion is a component important for the magnitude of the photocurrent. In this case, the exciton diffusion length must exceed the typical penetration depth of the light, so that the largest possible portion of the light can be used electrically. The excitons reach an interface by diffusion where electrons and holes are separated from one another. The material which absorbs the electrons is referred to as an acceptor, and the material which absorbs the holes is referred to as an electron donor.

A structure of a common organic solar cell which is already known from the literature consists of PIN or NIP diodes [Martin Pfeiffer, “Controlled doping of organic vacuum deposited dye layers: basics and applications,” PhD thesis, TU-Dresden, 1999, and WO 2011/161108 A1]: a pin solar cell in this case consists of a carrier/substrate with, attached to it, the usually transparent base contact, p-layer(s), i-layer(s), n-layer(s), and a cover contact. An NIP solar cell consists of a carrier/substrate with, attached to it, the usually transparent base contact, n-layer(s), i-layer(s), p-layer(s), and a cover contact. Here, n- or p-doping means that leads to an increase in the density of free electrons or holes in the thermal equilibrium state. Such layers are thus to be understood primarily as transport layers. It is also possible for n- or p-layer(s) to be at least partially nominally undoped and to preferably have n-conducting or p-conducting properties merely on the basis of the material properties (e.g., different mobility) or on the basis of different impurities (e.g., remaining residues from the synthesis or the layer production), or due to influences of the environment (e.g., adjacent layers, inward diffusion of metals or other organic materials, gas doping from the ambient atmosphere). In this sense, such layers are preferably to be understood as transport layers. The designation, i-layer, characterizes an undoped or intrinsic layer. One or more i-layers can consist here of one material (planar heterojunctions) as well as of a mixture of two or more materials (bulk heterojunctions) which have an inter-penetrating network.

Further known from the literature are organic PIN tandem cells and PIN multiple cells. WO 2011 161 108 A1 discloses a photoactive device with an electrode and a counter-electrode, wherein at least one organic layer system is arranged between the electrodes, and furthermore with at least two photoactive layer systems and, between the photoactive layer systems, at least two different transport layer systems of the same charge carrier type, characterized in that a transport layer system is adapted in terms of energy to one of the two photoactive layer systems, and the other transport layer system is transparent.

In an embodiment, the present invention provides a use of a compound according to embodiments of the invention in an organic electronic device—in particular, according to one of the exemplary embodiments described above. In particular, the advantages which have already been explained in conjunction with the compound of general formula (I) and the organic electronic device having such a compound are provided for the use of the compound according to embodiments of the invention in an organic electronic device.

In a preferred embodiment of the invention, the compound according to embodiments of the invention, and preferably several compounds according to embodiments of the invention, are used in an absorber layer of a solar cell.

The inventors have found that, due to the presence in particular of a further heterocyclic group, which can be a furan group or a thiophene group, as well as double bonds which are preferably arranged adjacently to at least one of the electron-withdrawing groups A1 and/or A2, but may also be arranged between a heterocyclic group and the central donor block Z, further molecules according to embodiments of the invention can be produced which have the already-mentioned advantageous properties.

Table 1 shows, in an overview, the structures, melting points, and absorption maxima (in nm and eV in solvent (LM)) of exemplary embodiments of compounds according to embodiments of the invention which fall under both the general formulas I and II.

TABLE 1

| | | M.p./ | λmax | |
|---|---|---|---|---|
| No. | Structure | ° C.^{a} | (LM)/nm^{b} | in eV |
| 11 | 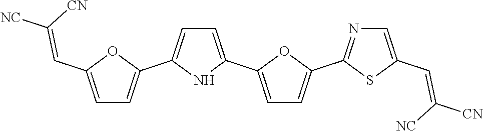 [SMILES: N#CC(C#N)=Cc1ccc(-c2ccc(-c3ccc(-c4ncc(C=C(C#N)C#N)s4)o3)[nH]2)o1] | 288 | 570 | 2.18 |
| 12 | 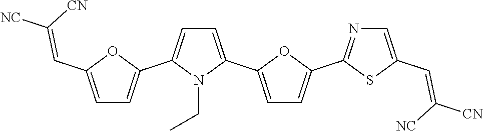 [SMILES: CCn1c(-c2ccc(C=C(C#N)C#N)o2)ccc1-c1ccc(-c2ncc(C=C(C#N)C#N)s2)o1] | 289 | 544 | 2.28 |
| 13 | 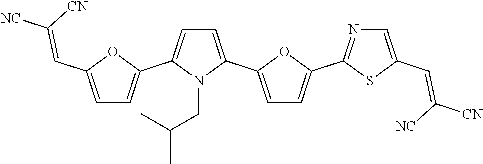 [SMILES: CC(C)Cn1c(-c2ccc(C=C(C#N)C#N)o2)ccc1-c1ccc(-c2ncc(C=C(C#N)C#N)s2)o1] | 270 | 542 | 2.29 |
| 14 | 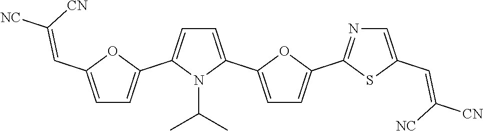 [SMILES: CC(C)n1c(-c2ccc(C=C(C#N)C#N)o2)ccc1-c1ccc(-c2ncc(C=C(C#N)C#N)s2)o1] | — ^{c} | 510 | 2.43 |
| 15 | 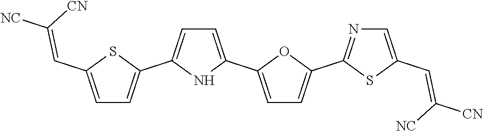 [SMILES: N#CC(C#N)=Cc1ccc(-c2ccc(-c3ccc(-c4ncc(C=C(C#N)C#N)s4)o3)[nH]2)s1] | — ^{c} | 545 | 2.27 |
| 16 | 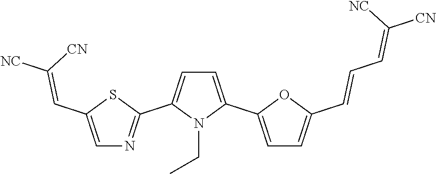 [SMILES: CCn1c(-c2ccc(/C=C/C=C(C#N)C#N)o2)ccc1-c1ncc(C=C(C#N)C#N)s1] | 256 | 543 | 2.28 |
| 17 | 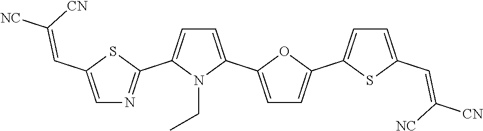 [SMILES: CCn1c(-c2ccc(-c3ccc(C=C(C#N)C#N)s3)o2)ccc1-c1ncc(C=C(C#N)C#N)s1] | — ^{d} | — ^{d} | — ^{d} |
| 18 | 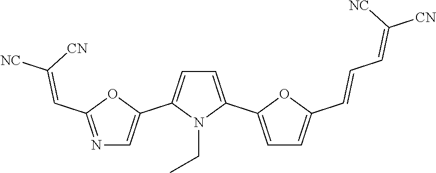 [SMILES: CCn1c(-c2ccc(/C=C/C=C(C#N)C#N)o2)ccc1-c1cnc(C=C(C#N)C#N)o1] | — ^{d} | — ^{d} | — ^{d} |
| 21 | 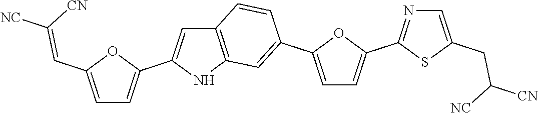 [SMILES: N#CC(C#N)=Cc1ccc(-c2cc3ccc(-c4ccc(-c5ncc(CC(C#N)C#N)s5)o4)cc3[nH]2)o1] | 307 | 529 | 2.34 |
| 22 | 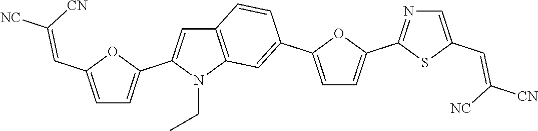 [SMILES: CCn1c(-c2ccc(C=C(C#N)C#N)o2)cc2ccc(-c3ccc(-c4ncc(C=C(C#N)C#N)s4)o3)cc21] | 352 | 530 | 2.34 |
| 23 | 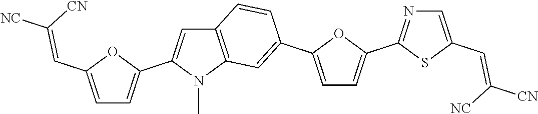 [SMILES: Cn1c(-c2ccc(C=C(C#N)C#N)o2)cc2ccc(-c3ccc(-c4ncc(C=C(C#N)C#N)s4)o3)cc21] | — ^{d} | — ^{d} | — ^{d} |
| 24 | 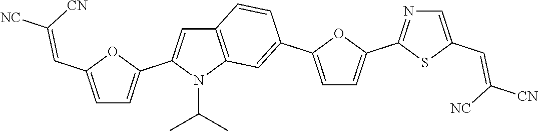 [SMILES: CC(C)n1c(-c2ccc(C=C(C#N)C#N)o2)cc2ccc(-c3ccc(-c4ncc(C=C(C#N)C#N)s4)o3)cc21] | — ^{d} | — ^{d} | — ^{d} |
| 25 | 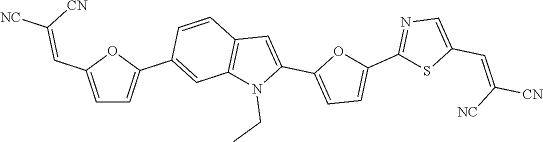 [SMILES: CCn1c(-c2ccc(-c3ncc(C=C(C#N)C#N)s3)o2)cc2ccc(-c3ccc(C=C(C#N)C#N)o3)cc21] | — ^{d} | — ^{d} | — ^{d} |
| 31 | 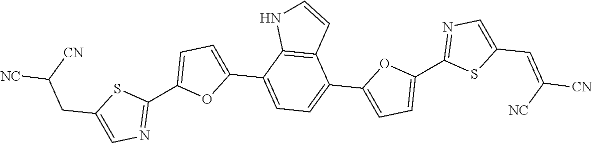 [SMILES: N#CC(C#N)=Cc1cnc(-c2ccc(-c3ccc(-c4ccc(-c5ncc(CC(C#N)C#N)s5)o4)c4[nH]ccc34)o2)s1] | — ^{c} | 511 | 2.42 |
| 32 | 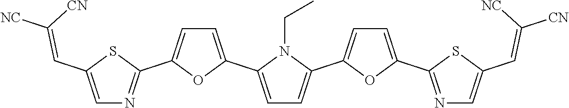 [SMILES: CCn1c(-c2ccc(-c3ncc(C=C(C#N)C#N)s3)o2)ccc1-c1ccc(-c2ncc(C=C(C#N)C#N)s2)o1] | 320 | 543 | 2.28 |
| 33 | 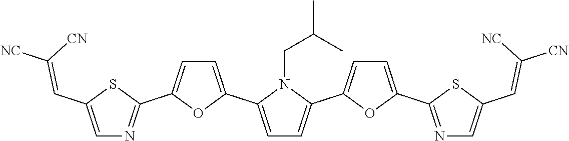 [SMILES: CC(C)Cn1c(-c2ccc(-c3ncc(C=C(C#N)C#N)s3)o2)ccc1-c1ccc(-c2ncc(C=C(C#N)C#N)s2)o1] | 291 | 540 | 2.30 |
| 34 | 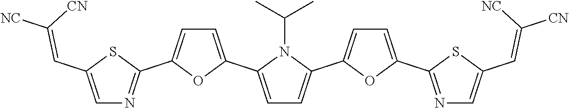 [SMILES: CC(C)n1c(-c2ccc(-c3ncc(C=C(C#N)C#N)s3)o2)ccc1-c1ccc(-c2ncc(C=C(C#N)C#N)s2)o1] | 332 | 522 | 2.38 |
| 35 | 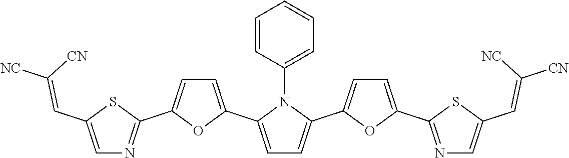 [SMILES: N#CC(C#N)=Cc1cnc(-c2ccc(-c3ccc(-c4ccc(-c5ncc(C=C(C#N)C#N)s5)o4)n3-c3ccccc3)o2)s1] | 323 | 573 | 2.16 |

^{a}onset DSC (dynamic differential calorimetry; start of the melting range; extrapolated start temperature (intersection point inflectional tangent baseline)
^{b}in dichloromethane unless otherwise noted
^{c} no melting peak in DSC
^{d} data not available yet

Surprisingly, it has been found that the compounds according to embodiments of the invention have particularly strong absorption (i.e., high optical density at the absorption maximum or high integral over the optical density in the visible spectral range compared to similar compounds outside the range described here).

Table 2 below shows various parameters of compounds according to embodiments of the invention in direct comparison. The photovoltaic parameters V_{oc}, J_{sc}, and FF are each based upon solar cells with a 30 nm thick mixed layer formed from the respective donor material of these compounds and fullerene C60 as photoactive layer on glass with the structure ITO/C60 (15 nm)/the respective compounds: C60 (30 nm)/NHT169 or NHT049 (10 nm)/NHT169 or NHT049:NDP9 (30 nm)/NDP9 (1 nm)/Au (50 nm), measured under AM1.5 illumination (AM=air mass; AM=1.5 in this spectrum, the global radiation power is 1,000 W/m²; AM=1.5 as the standard value for the measurement of solar modules). Table 2 shows in particular the optical density at the absorption maximum (ODmax), the optical integral in the visible range (GD integral), and V_{oc}, J_{sc}, FF, and the efficiency.

TABLE 2

| | | OD | | | | |
|---|---|---|---|---|---|---|
| | | integral | | | | |
| | | (400- | | | | |
| | | 900 | | J_{sc} | | |
| | | nm) | V_{oc} | [mA/ | FF | eff |
| No. | Structure | [nm] | [V] | cm²] | [%] | [%] |
| 11 | 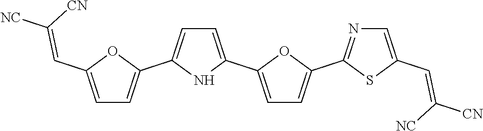 [SMILES: N#CC(C#N)=Cc1ccc(-c2ccc(-c3ccc(-c4ncc(C=C(C#N)C#N)s4)o3)[nH]2)o1] | 88 | 0.97 | 5.2 | 74.5 | 3.8 |
| 12 | 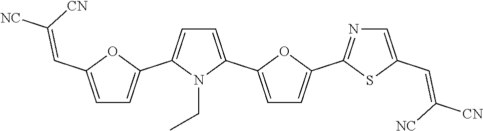 [SMILES: CCn1c(-c2ccc(C=C(C#N)C#N)o2)ccc1-c1ccc(-c2ncc(C=C(C#N)C#N)s2)o1] | 72 | 0.99 | 11.5 | 69.5 | 7.9 |
| 13 | 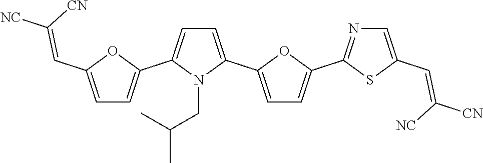 [SMILES: CC(C)Cn1c(-c2ccc(C=C(C#N)C#N)o2)ccc1-c1ccc(-c2ncc(C=C(C#N)C#N)s2)o1] | 110 | 0.98 | 9.5 | 49.5 | 4.6 |
| 14 | 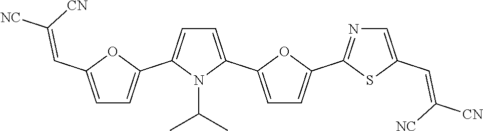 [SMILES: CC(C)n1c(-c2ccc(C=C(C#N)C#N)o2)ccc1-c1ccc(-c2ncc(C=C(C#N)C#N)s2)o1] | 37 | — ^{b} | — ^{b} | — ^{b} | — ^{b} |
| 15 | 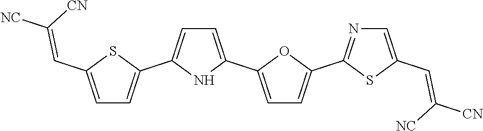 [SMILES: N#CC(C#N)=Cc1ccc(-c2ccc(-c3ccc(-c4ncc(C=C(C#N)C#N)s4)o3)[nH]2)s1] | 92 | — ^{b} | — ^{b} | — ^{b} | — ^{b} |
| 16 | 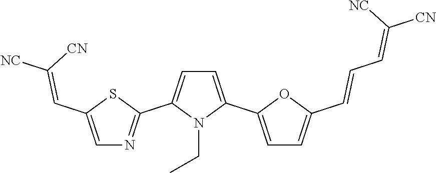 [SMILES: CCn1c(-c2ccc(/C=C/C=C(C#N)C#N)o2)ccc1-c1ncc(C=C(C#N)C#N)s1] | 107 | 1.02 | 10.7 | 60.7 | 6.6 |
| 17 | 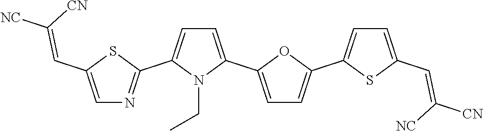 [SMILES: CCn1c(-c2ccc(-c3ccc(C=C(C#N)C#N)s3)o2)ccc1-c1ncc(C=C(C#N)C#N)s1] | | | | | |
| 18 | 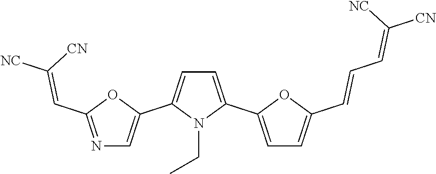 [SMILES: CCn1c(-c2ccc(/C=C/C=C(C#N)C#N)o2)ccc1-c1cnc(C=C(C#N)C#N)o1] | | | | | |
| 21 | 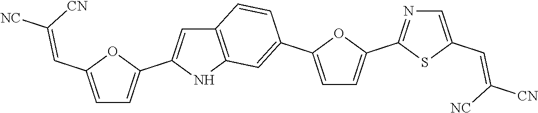 [SMILES: N#CC(C#N)=Cc1ccc(-c2cc3ccc(-c4ccc(-c5ncc(C=C(C#N)C#N)s5)o4)cc3[nH]2)o1] | 98 | — ^{b} | — ^{b} | — ^{b} | — ^{b} |
| 22 | 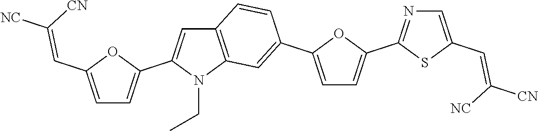 [SMILES: CCn1c(-c2ccc(C=C(C#N)C#N)o2)cc2ccc(-c3ccc(-c4ncc(C=C(C#N)C#N)s4)o3)cc21] | 79 | 0.99 | 10.5 | 74.0 | 7.7 |
| 23 | 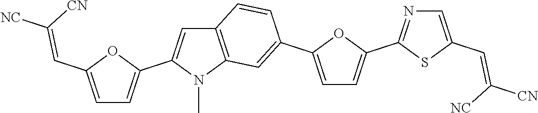 [SMILES: Cn1c(-c2ccc(C=C(C#N)C#N)o2)cc2ccc(-c3ccc(-c4ncc(C=C(C#N)C#N)s4)o3)cc21] | | | | | |
| 24 | 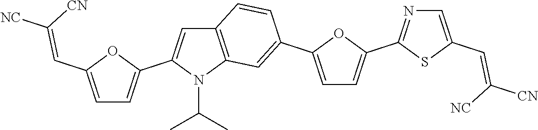 [SMILES: CC(C)n1c(-c2ccc(C=C(C#N)C#N)o2)cc2ccc(-c3ccc(-c4ncc(C=C(C#N)C#N)s4)o3)cc21] | | | | | |
| 25 | 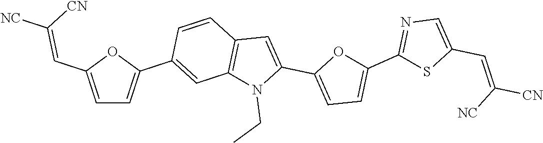 [SMILES: CCn1c(-c2ccc(-c3ncc(C=C(C#N)C#N)s3)o2)cc2ccc(-c3ccc(C=C(C#N)C#N)o3)cc21] | | | | | |
| 31 | 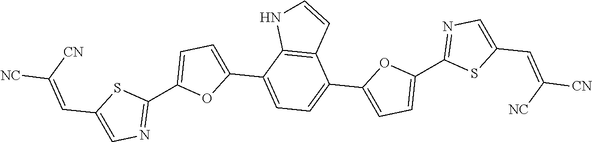 [SMILES: N#CC(C#N)=Cc1cnc(-c2ccc(-c3ccc(-c4ccc(-c5ncc(C=C(C#N)C#N)s5)o4)c4[nH]ccc34)o2)s1] | 55 | — ^{b} | — ^{b} | — ^{b} | — ^{b} |
| 32 | 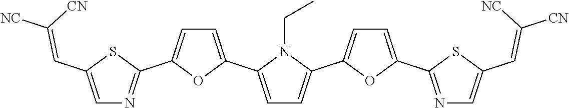 [SMILES: CCn1c(-c2ccc(-c3ncc(C=C(C#N)C#N)s3)o2)ccc1-c1ccc(-c2ncc(C=C(C#N)C#N)s2)o1] | 87 | 0.94 | 12.8 | 71.2 | 8.6 |
| 33 | 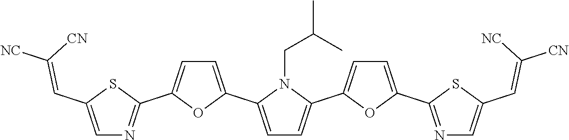 [SMILES: CC(C)Cn1c(-c2ccc(-c3ncc(C=C(C#N)C#N)s3)o2)ccc1-c1ccc(-c2ncc(C=C(C#N)C#N)s2)o1] | 85 | 0.95 | 12.4 | 72.4 | 8.5 |
| 34 | 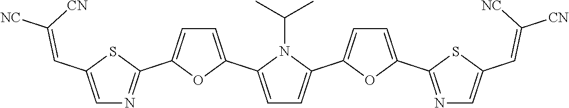 [SMILES: CC(C)n1c(-c2ccc(-c3ncc(C=C(C#N)C#N)s3)o2)ccc1-c1ccc(-c2ncc(C=C(C#N)C#N)s2)o1] | 97 | — ^{b} | — ^{b} | — ^{b} | — ^{b} |
| 35 | 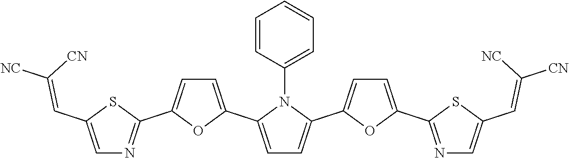 [SMILES: N#CC(C#N)=Cc1cnc(-c2ccc(-c3ccc(-c4ccc(-c5ncc(C=C(C#N)C#N)s5)o4)n3-c3ccccc3)o2)s1] | 101 | 0.97 | 13.3 | 70.6 | 9.1 |

^{b} cell not measured

The spectral data relate to vacuum vapor layers on quartz glass that are 30 nm thick.

It was also shown that many derivatives of the compounds according to embodiments of the invention not only absorb light, but can also be evaporated without residue in a vacuum. High photocurrents can be produced due to very good charge transport properties and good absorption properties. Thus, very well combined tandem/triple/quadruple/or multi-junction solar cells can be produced.

Embodiments of the invention are explained in greater detail below with reference to the drawings.

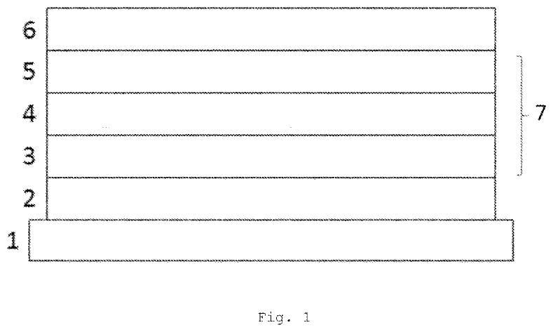

FIG. 1 shows a schematic illustration of an exemplary embodiment of an organic electronic device in cross-section.

The organic electronic device according to an embodiment of the invention has a layer system 7, wherein at least one layer of the layer system 7 has a compound of the general formula I according to embodiments of the invention.

The organic electronic device comprises a first electrode 2, a second electrode 6, and a layer system 7, wherein the layer system 7 is arranged between the first electrode 2 and the second electrode 6. Here, at least one layer of the layer system 7 has at least one compound of the general formula I according to embodiments of the invention.

The layer system 7 has a photoactive layer 4, and preferably a light-absorbing photoactive layer 4, wherein the photoactive layer 4 comprises the at least one compound according to embodiments of the invention.

In one exemplary embodiment, the organic solar cell has a substrate 1, e.g., made of glass, on which an electrode 2 is located, which, for example, comprises ITO. Thereupon is arranged the layer system 7 having an electron-transporting layer 3 (ETL) and a photoactive layer 4 having at least one compound according to embodiments of the invention, a p-conducting donor material, and an n-conducting acceptor material, e.g., C60 fullerene, either as a planar heterojunction or as a bulk heterojunction. Thereover are arranged a p-doped hole transport layer 5 (HTL), and an electrode 6 made of gold or aluminum.

The layer system, and in particular individual layers, of the device can be produced by evaporation of the compounds in a vacuum, with or without carrier gas, or processing of a solution or suspension—for example, during coating or printing. Individual layers can also be applied by sputtering. The production of the layers by evaporation in a vacuum is advantageous, wherein the carrier substrate may be heated. The organic materials are in this case printed onto the foils, bonded, coated, applied by vapor deposition, or otherwise applied in the form of thin films or small volumes. All methods which are also used for electronics on glass, ceramic, or semiconductive carriers are also suitable for the production of the thin layers.

The chemical compound of the general formula I has the following structure:

A1-(T1)ₐ-(T2)_{b}-(Z)_{c}-(T3)_{d}-(T4)ₑ-A2 (I)
with the parameters a, b, d, e each independently 0 or 1, with the proviso that at least one of the parameters a, b, d, e=1,
with the parameter c=1, 2, or 3,
wherein the general group Z is a block of two groups M and N, linked as *-M-N—* or *—N-M-*,
wherein * denotes the attachment to the groups T1, T2, T3, T4, A1, and A2,
wherein the groups M are each independently selected from:

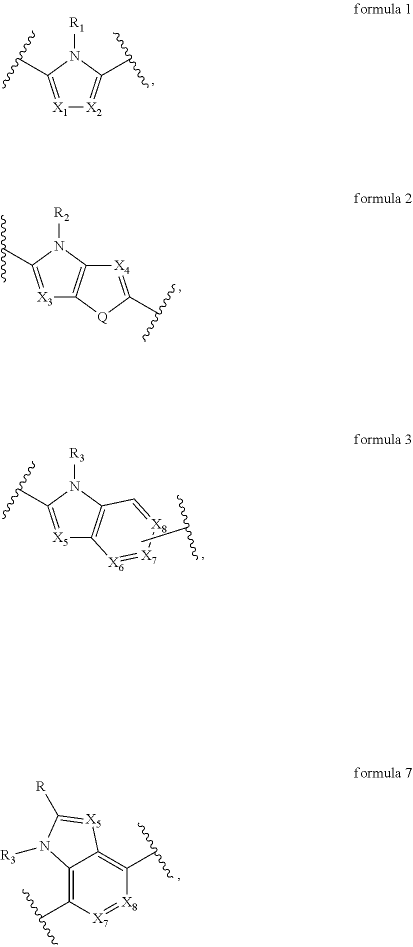

wherein the groups N are each independently selected from:

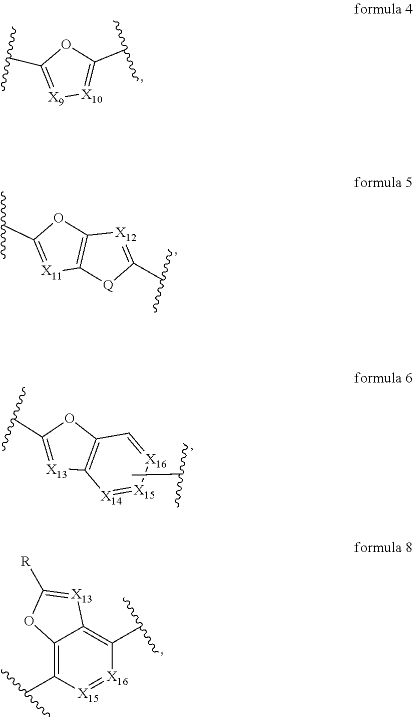

wherein M and N are each linked such that at least one N atom of the group M and an O atom of the group N are each bonded to one another via 2 C atoms, and [symbol] denotes the attachment to the other groups in the compound of the general formula I,
with X₁-X₁₆ independently selected from N or C—R, with the proviso that, in each of the groups of the formulas 3 and 6, a group from the groups X₈/X₇ and X₁₆/X₁₅ denotes the attachment [symbol] to the other groups in the compound of the general formula I,
with R in each case independently selected from the group consisting of H, halogen, branched or linear, cyclic or open-chain C1-C10 alkyl, wherein H atoms of the C1-C10 alkyl can be substituted, and C atoms of the C1-C10 alkyl can be substituted by heteroatoms, C2-C10 alkenyl, O-alkyl, S-alkyl, O-alkenyl, S-alkenyl, alkynyl, aryl, heteroaryl, wherein, in all of these groups, H atoms can be substituted, CN, NR10R11, with R10 and R11 each independently selected from H, branched or linear, cyclic or open-chain C1-C10 alkyl, wherein H atoms of the C1-C10 alkyl can be substituted, and C atoms of the C₁-C₁₀ alkyl can be substituted by heteroatoms,
with R₁ to R₃ each independently selected from the group consisting of H, branched or linear, cyclic or open-chain C₁-C₁₀ alkyl, wherein H atoms of the C₁-C₁₀ alkyl can be substituted, and C atoms of the C₁-C₁₀ alkyl can be substituted by heteroatoms, substituted or unsubstituted C₂-C₁₀ alkenyl, substituted or unsubstituted aryl, substituted or unsubstituted heteroaryl, CN,
wherein the electron-withdrawing groups A1 and A2 are, independently, electron-withdrawing groups having at least one C═C double bond,
wherein the groups T1, T2, T3, and T4 are each independently selected from:

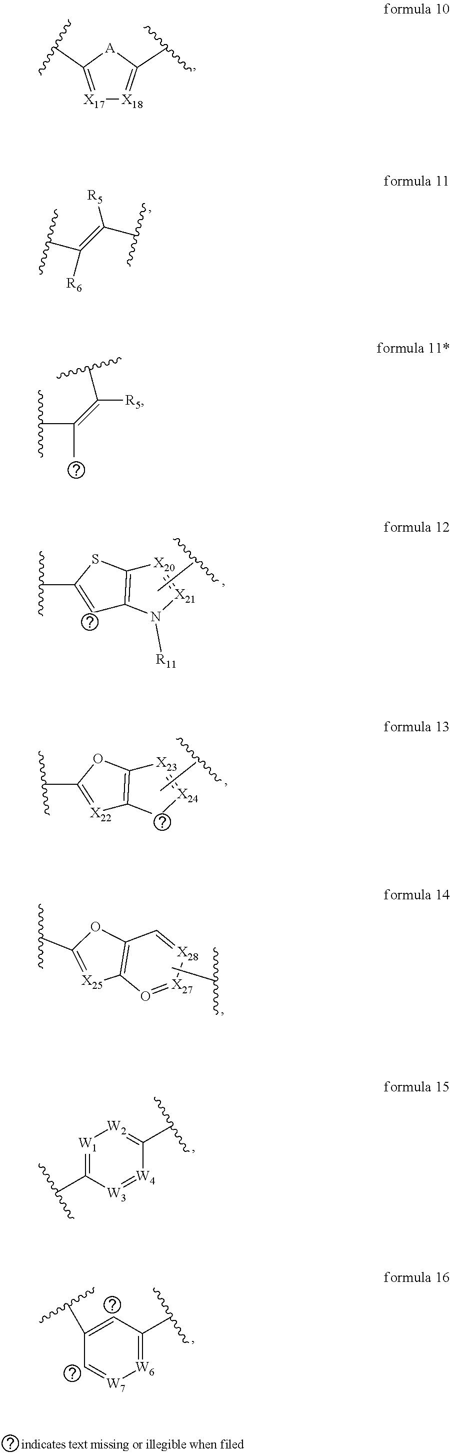

wherein [symbol] denotes the attachment to the other groups in the compound of the general formula I, with the proviso that at least one of the groups T1 to T4 is the formula 10,
with R₅ and R₆ each independently selected from the group consisting of H, CN, F, aryl, heteroaryl, C₂-C₁₀ alkenyl, alkynyl, branched or linear, cyclic or open-chain C₁-C₁₀ alkyl, wherein H atoms of the C₁-C₁₀ alkyl can be substituted, wherein, if the substituent R₁₃ is present in the compound of the formula I, a ring closure between R₅ with R₁₃ or R₆ with R₁₃ is possible, with the proviso that, between R₅ and R₁₃ or between R₆ and R₁₃, in each case there is located the double bond from formula 11,
with W₁ to W₈ each independently selected from N, CR, wherein R is defined as described above,
with X₁₇ and X₁₈ independently selected from N and C—R, wherein R is defined as described above, with the proviso that at least X₁₇ or X₁₈ is N,
with X₁₉-X₂₇ independently selected from N and C—R, wherein R is defined as described above, and with the proviso that, in the groups of the formulas 12, 13, and 14, a group each from the groups X₂₀/X₂₁, X₂₃/X₂₄, and X₂₆/X₂₇ denotes the attachment [symbol] to the other groups in the compound of the general formula I,
with A selected from the group consisting of S, O, NR₉, and Se,
with Q selected from the group consisting of S, O, NR₉, and Se,
wherein, for the groups A and Q, the substituent R₉ is in each case independently selected from H, CN, branched or linear, cyclic or open-chain C₁-C₁₀ alkyl, wherein the H atoms of the C₁-C₁₀ alkyl may be substituted, C₂-C₁₀ alkenyl, O-alkyl, S-alkyl, O-alkenyl, S-alkenyl, alkynyl, substituted or unsubstituted aryl, substituted or unsubstituted heteroaryl.

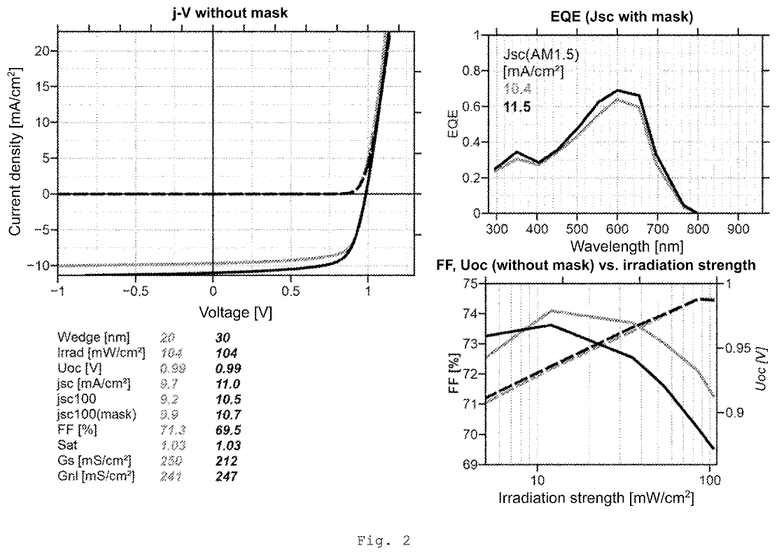

FIG. 2 shows a current-voltage curve of an organic electronic device having the compound 12.

FIG. 2 shows the current-voltage curve of a BHJ cell with the structure: ITO/C60 (15 nm)/compound 12: C60 (30 nm, 2:1, 50° C.)/NHT169 (10 nm)/NHT169:NDP9 (30 nm, 9.1 wt %)/NDP9 (1 nm)/Au (50 nm), wherein the photoactive layer 4 is a bulk heterojunction (BHJ). Here, ITO is indium tin oxide, NDP9 is a commercial p-dopant of Novaled GmbH, and NHT169 is a commercial hole conductor of Novaled GmbH. In this exemplary embodiment, the organic electronic device is a solar cell.

In the solar cell with the compound (12), the fill factor FF is 69.5%, the open-circuit voltage Uoc is 0.99 V, and the short-circuit current Jsc is 11.5 mA/cm². The cell efficiency of such an organic electronic device, and in particular a solar cell, having the compound (12) is 7.91%.

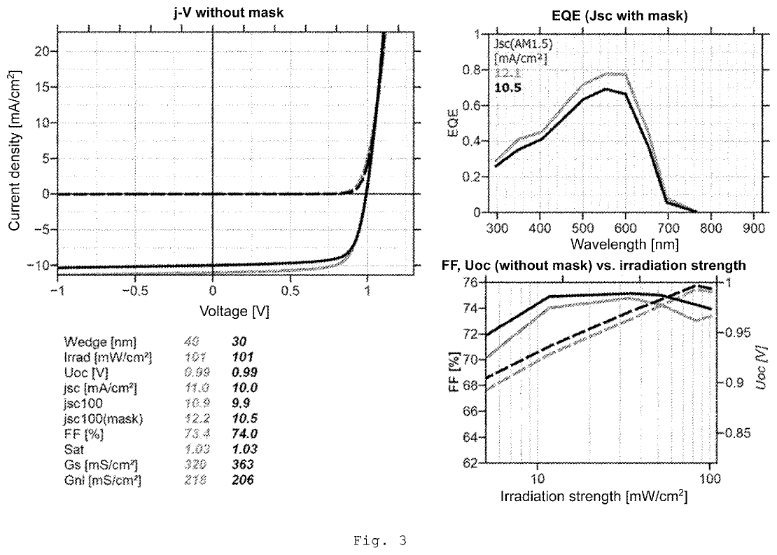

FIG. 3 shows a current-voltage curve of an organic electronic device having the compound 22.

FIG. 3 shows the current-voltage curve of a BHJ cell with the structure: ITO/C60 (15 nm)/compound 22: C60 (30 nm, 3:2, 50° C.)/NHT049 (10 nm)/NHT049:NDP9 (30 nm, 9.7 wt %)/NDP9 (1 nm)/Au (50 nm), wherein the photoactive layer 4 is a bulk heterojunction (BHJ). Here, ITO is indium tin oxide, NDP9 is a commercial p-dopant of Novaled GmbH, and NHT049 is a commercial hole conductor of Novaled GmbH. In this exemplary embodiment, the organic electronic device is a solar cell.

In the solar cell with the compound (22), the fill factor FF is 74.0%, the open-circuit voltage Uoc is 0.99 V, and the short-circuit current Jsc is 10.5 mA/cm². The cell efficiency of such an organic electronic device, and in particular a solar cell, having the compound (22) is 7.69%.

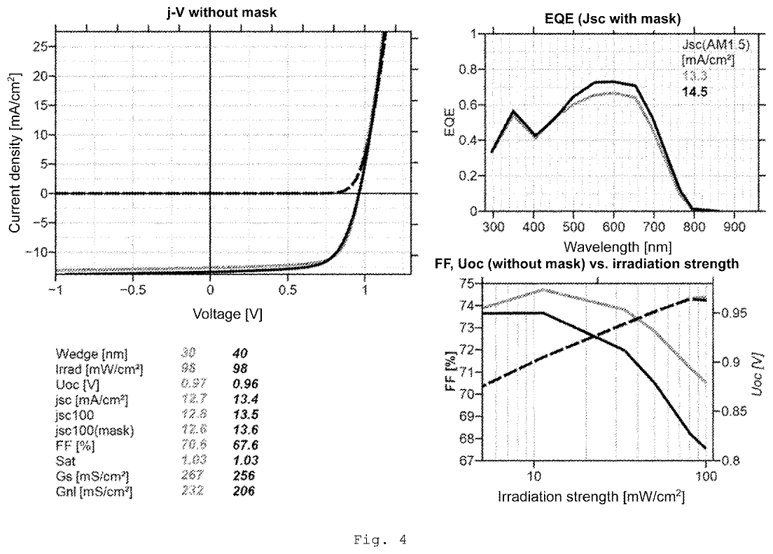

FIG. 4 shows a current-voltage curve of an organic electronic device having the compound 35.

FIG. 4 shows the current-voltage curve of a BHJ cell with the structure: ITO/C60 (15 nm)/compound 35: C60 (30 nm, 2:1, 50° C.)/NHT049 (10 nm)/NHT049:NDP9 (30 nm, 9.7 wt %)/NDP9 (1 nm)/Au (50 nm), wherein the photoactive layer is a bulk heterojunction (BHJ). Here, ITO is indium tin oxide, NDP9 is a commercial p-dopant of Novaled GmbH, and NHT049 is a commercial hole conductor of Novaled GmbH. In this exemplary embodiment, the organic electronic device is a solar cell.

In the solar cell having the compound (35), the fill factor FF is 67.6%, the open-circuit voltage Uoc is 0.96 V, and the short-circuit current Jsc is 14.5 mA/cm². The cell efficiency of such an organic electronic device, and in particular a solar cell, having the compound (35) is 9.40%.

In the following, syntheses of specific exemplary embodiments are shown by way of example.

The general compound (I) can be synthesized by one of the methods described below. This is intended here to be an exemplary representation and can be varied in the sequence of its individual steps, or can be modified by other known methods. A combination of individual reaction steps or a change in parts of the synthesis route is also possible.

### Synthesis of {[2-(5-{5-[5-(2,2-dicyanoethenyl)furan-2-yl]-1-ethyl-1H-pyrrol-2-yl}furan-2-yl)-1,3-thiazol-5-yl]methylidene}propanedinitriles (compound 12)

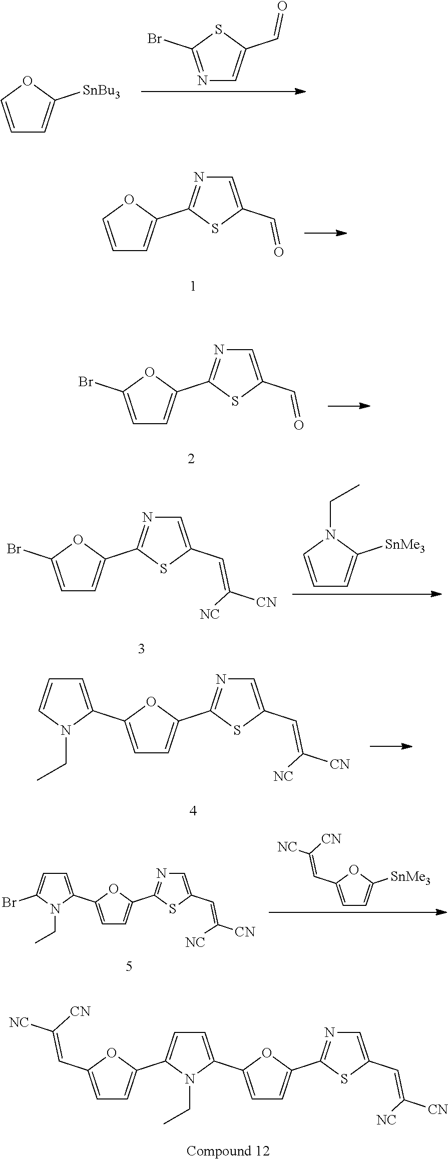

### Synthesis of 2-(furan-2-yl)-1,3-thiazole-5-carbaldehydes (1)

4.9 g (25.5 mmol) of 2-bromothiazole-5-carboxaldehyde and 9.4 g (25.5 mmol) of 2-(tributylstannyl)furan were dissolved in 51 mL of toluene, and the reaction mixture was degassed. 1.55 g (1.28 mmol) of tetrakis-(triphenylphosphine) palladium (0) was added, and the reaction mixture was heated to boiling overnight. After the reaction mixture had been cooled to room temperature, it was mixed with DCM. The organic phase was washed with water and sat. NaCl solution. Crude product was purified by silica gel filtration in DCM, in which 2.9 g (64% yield) of clean 1 was isolated. 1H-NMR in d6-acetone, ppm: 9.98 (s, 1H), 8.48 (s, 1H), 7.75 (dd, 1H), 7.20 (dd, 1H), 6.63 (dd, 1H).

### Synthesis of 2-(5-bromofuran-2-yl)-1,3-thiazole-5-carbaldehydes (2)

7.21 g (40.2 mmol) of 1 was dissolved in 40 ml of DMF under argon. 8.03 g (44.2 mmol) of NBS was added in small portions, and the reaction mixture was stirred at room temperature for 3 h. The mixture was put on ice. The precipitate formed was filtered, washed with water and ethanol, and dried in air. Here, 9.82 g (95% yield) of 2 was isolated, without further purification. 1H-NMR in d6-acetone, ppm: 10.13 (s, 1H), 8.63 (s, 1H), 7.36 (d, 1H), 6.85 (d, 1H).

### Synthesis of {[2-(5-bromofuran-2-yl)-1,3-thiazol-5-yl]methylidene}propanedinitriles (3)

5.24 g (20.3 mmol) of 2 was dissolved in 15 ml of ethanol. 139 mg (2.03 mmol) of 8-alanine and 1.31 g (26.4 mmol) of malodinitrile were added. The reaction mixture was stirred overnight at room temperature. The precipitate formed was filtered, washed with ethanol, and dried in air. Here, 5.57 g (90% yield) of 3 was isolated, without further purification. 1H-NMR in d6-acetone, ppm: 8.50 (s, 1H), 8.42 (s, 1H), 7.32 (d, 1H), 6.86 (d, 1H).

### Synthesis of ({2-[5-(1-ethyl-1H-pyrrol-2-yl)furan-2-yl]-1,3-thiazol-5-yl}methylidene)propanedinitriles (4)

1.55 g (6 mmol) of 1-ethyl-2-(trimethylstannyl)-1H-pyrroles and 2.2 g (7.2 mmol) of 3 were dissolved in 11 ml of dioxane, and the solution was degassed. 0.15 g (0.3 mmol) of bis (tri-tert.-butylphosphine) palladium (0) was added, and the reaction mixture was stirred at 60° C. overnight. After the reaction mixture had been cooled to room temperature, it was mixed with DCM. The organic phase was washed with water. The crude product was recrystallized from ethanol; in this case 0.63 g of 4 was isolated. 1H-NMR in d6-acetone, ppm: 8.58 (s, 1H), 8.54 (s, 1H), 7.53 (d, 1H), 7.05 (dd, 1H), 6.82 (d, 1H), 6.73 (dd, 1H), 6.19 (dd, 1H), 4.43 (qa, 2H), 1.43 (t, 3H).

### Synthesis of ({2-[5-(5-bromo-1-ethyl-1H-pyrrol-2-yl)furan-2-yl]-1,3-thiazol-5-yl}methylidene)propanedinitriles (5)

352 mg (1.1 mmol) of 4 was dissolved in 17 ml of DMF. 186 mg (1.04 mmol) of NBS was added, and the reaction mixture was stirred overnight at room temperature. Subsequently, the mixture was added to ice. The precipitate formed was filtered, washed with water and ethanol, and dried in air. Here, 356 mg (81% yield) of 5 was isolated, without further purification. 1H-NMR in d6-acetone, ppm: 8.59 (s, 1H), 8.56 (s, 1H), 7.53 (d, 1H), 6.91 (dd, 1H), 6.75 (d, 1H), 6.34 (d, 1H), 4.38 (qa, 2H), 1.46 (t, 3H).

Synthesis of {[2-(5-{5-[5-(2,2-dicyanoethenyl)furan-2-yl]-1-ethyl-1H-pyrrol-2-yl}furan-2-yl)-1,3-thiazol-5-yl]methylidene}propanedinitriles (compound 12) 226 mg (0.57 mmol) of 5 and 208 mg (0.68 mmol) of 5-(trimethylstannyl)-2-dicyanovinylfuran were dissolved in 1.7 ml of dioxane. The reaction mixture was degassed, and 7.23 mg (0.0143 mmol) of bis-(tri-tert.-butylphosphine) palladium (0) were added. The mixture was heated overnight at 80° C. The reaction mixture was cooled to room temperature, and the precipitate was filtered. The crude product was washed with methanol and recrystallized from acetonitrile. Here, 60 mg (23% yield, HPLC purity 97.5% at 481 nm) of compound 12 were isolated.

### Synthesis of {[2-(5-{2-[5-(2,2-dicyanoethenyl)furan-2-yl]-1-ethyl-1H-indol-6-yl}furan-2-yl)-1,3-thiazol-5-yl]methylidene}propanedinitriles (compound 22)

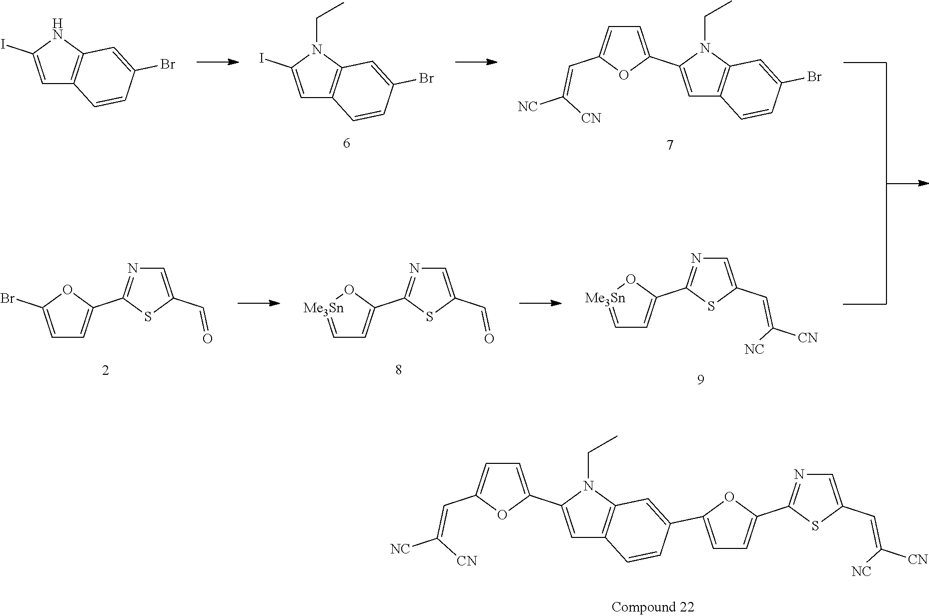

### Synthesis of 6-bromo-1-ethyl-2-iodo-1H-indoles (6)

5 g (15.5 mmol) of 6-bromo-1-ethyl-2-iodo-1H-indoles were dissolved in 155 mL of DMF. 2.59 g (23.3 mmol) of ethyl bromide and 4.33 g (31 mmol) of potassium carbonate were added, and the mixture was stirred overnight at room temperature. Subsequently, the mixture was mixed with water and extracted with ethyl acetate. Organic phase was washed with sat. NaCl and with water. Crude product was purified by silica gel filtration. Here, 4.71 g (87% yield) of 6 were isolated. 1H-NMR in d6-acetone, ppm: 7.69 (d, 1H), 7.42 (d, 1H), 7.11 (dd, 1H), 6.78 (s, 1H), 4.29 (qa, 2H), 1.27 (t, 3H).

### Synthesis of {[5-(6-bromo-1-ethyl-1H-indol-2-yl)furan-2-yl]methylidene}propanedinitriles (7)

4.72 g (13.5 mmol) of 6 and 4.14 g (13.5 mmol) of 5-(trimethylstannyl)-2-dicyanovinyl furan were dissolved in dioxane, and the mixture was degassed. 0.234 g (0.34 mmol) (3-chloropyridyl)-(1,3-diisopropylimidazol-2-ylidene) palladium(II)dichloride and 0.256 g (1.69 mmol) of cesium fluoride were added. The reaction mixture was stirred overnight at 80° C. Subsequently, the mixture was mixed with DCM and washed with water. The crude product was mixed with ethanol and stirred at room temperature for 1 h. The solid was filtered off, washed with ethanol, and dried. Here, 1.71 g (35% yield) of 7 were isolated. 1H-NMR in d6-acetone, ppm: 8.06 (s, 1H), 7.85 (m, 1H), 7.64 (m, 2H), 7.36 (d, 1H), 7.29 (m, 2H), 4.72 (qa, 2H), 1.42 (t, 3H).

### Synthesis of 2-[5-(trimethylstannyl)furan-2-yl]-1,3-thiazole-5-carbaldehydes (8)

1.52 g (15.1 mmol) of 1-methylpiperazine was dissolved in 50 ml of dry THE and cooled to −78° C. under argon. 6 ml (15.1 mmol) of 2.5-M-nBuLi was slowly added, and the mixture was stirred for 15 min at −78° C. The solution of 3.54 g (13.7 mmol) of 2 was added to 18 mL of dry THE at −78° C., and the mixture was stirred at −78° C. for a further 15 min. 1.91 g (16.4 mmol) of TMEDA and 6.58 ml (16.4 mmol) of 2,5-M-nBuLi were added in succession, and the reaction mixture was stirred at −78° C. for 2 h. Subsequently, 16.4 ml (16.4 mmol) of 1-M-Me3SnCl solution was added, and the mixture was stirred overnight at room temperature. The reaction mixture was mixed with sat. NaCl solution and extracted with MTBE. Organic Phase was washed with water and dried over sodium sulfate. The crude product 8, 4.3 g (92% yield) was reacted further without further purification. 1H-NMR in d6-acetone, ppm: 10.05 (s, 1H), 8.56 (s, 1H), 7.25 (d, 1H), 6.88 (d, 1H), 0.38 (s, 9H).

### Synthesis of (({2-[5-(trimethylstannyl)furan-2-yl]-1,3-thiazol-5-yl}methylidene)propanedinitriles (9)

17 g (49.7 mmol) of 8 was dissolved in 48 ml of ethanol. 4.27 g (64.6 mmol) of malodinitrile and 0.45 g (4.96 mmol) of ß-alanine were added. The reaction mixture was stirred overnight at room temperature. The precipitate was filtered off, washed with ethanol, and dried. Here, 12.5 g (65% yield) of 9 were isolated. 1H-NMR in d6-acetone, ppm: 8.47 (s, 1H), 8.41 (s, 1H), 7.27 (d, 1H), 6.85 (d, 1H), 0.33 (s, 9H).

### Synthesis of {[2-(5-{2-[5-(2,2-dicyanoethenyl)furan-2-yl]-1-ethyl-1H-indol-6-yl}furan-2-yl)-1,3-thiazol-5-yl]methylidene}propanedinitriles (compound 22)

549 mg (1.5 mmol) of 7 and 585 mg (1.5 mmol) of 9 were dissolved in 9 ml of dioxane, and the solution was degassed. 19.2 mg (0.04 mmol) of bis (tri-tert.-butylphosphine) palladium (0) was added, and the reaction mixture was stirred overnight at 60° C. The precipitate was filtered off, washed with methanol, and recrystallized twice from chlorobenzene. Here, 222 mg (29% yield, HPLC purity 94.5% at 530 nm) of compound 22 were isolated.

### Synthesis of 2,2′-{(1-phenyl-1H-pyrrole-2,5-diyl)bis[(furan-5,2-diyl)-1,3-thiazole-2,5-diylmethanylylidene]}dipropanedinitriles (compound 35)

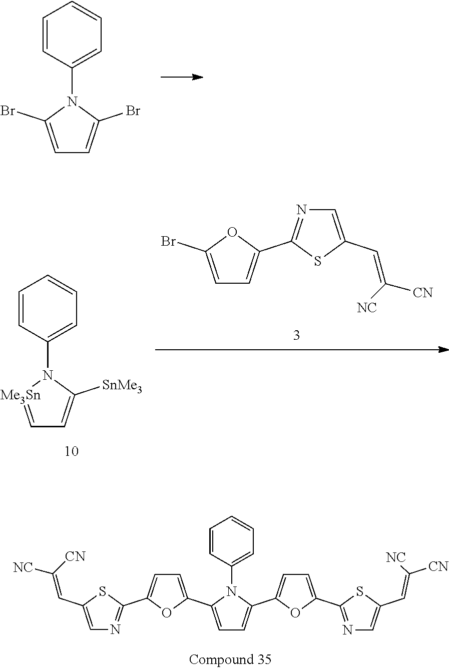

### Synthesis of 1-phenyl-2,5-bis(trimethylstannyl)-1H-pyrroles (10)

3.49 g (11.6 mmol) of 2,5-dibromo-1-phenyl-1H-pyrroles was dissolved in 105 ml of dry THF, and the solution was cooled to −78° C. 54.6 ml (92.8 mmol) of 1.7-M-tBuLi was slowly added dropwise at −78° C., and the mixture was stirred at −78° C. for 3 h. Subsequently, 92.8 ml (92.8 mmol) of 1-M-Me3SnCl solution was added, and the reaction mixture was stirred overnight at room temperature. The reaction mixture was mixed with petroleum ether and extracted with water. Organic phase was dried over sodium sulfate. Crude product 10 was recrystallized from ethanol. Here, 1.48 g (27% yield) of 10 were isolated. 1H-NMR in d6-acetone, ppm: 7.54 (m, 3H), 7.32 (m, 2H), 6.45 (s, 2H), 0.07 (s, 18H).

### Synthesis of 2,2′-{(1-phenyl-1H-pyrrole-2,5-diyl)bis[(furan-5,2-diyl)-1,3-thiazole-2,5-diylmethanylylidene]}dipropanedinitriles (compound 35)

469 mg (1.0 mmol) of 10 and 765 mg (2.5 mmol) of 3 were dissolved in 5 ml of dioxane, and the solution was degassed. 12.8 mg (0.025 mmol) of bis (tri-tert.-butylphosphine) palladium (0) was added, and the reaction mixture was stirred overnight at 80° C. The precipitate was filtered off, washed with ethanol, and recrystallized twice from chlorobenzene. Here, 210 mg (35% yield, HPLC purity 99.3% at 573 nm) of compound 35 were isolated. While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A compound of the general formula I
A1-(T1)ₐ-(T2)_{b}-(Z)_{c}-(T3)_{d}-(T4)ₑ-A2 (I),
wherein the parameters a, b, d, and e are each independently 0 or 1,
wherein at least one of the parameters a, b, d, and e is equal to =1,
wherein the parameter c is equal to 1, 2, or 3,
wherein the general group Z is a block of two groups M and N, linked as *-M-N—* or *—N-M-*, wherein * denotes an attachment to groups T1, T2, T3, T4, A1, and A2,
wherein the groups M are each independently selected from the following formulas: 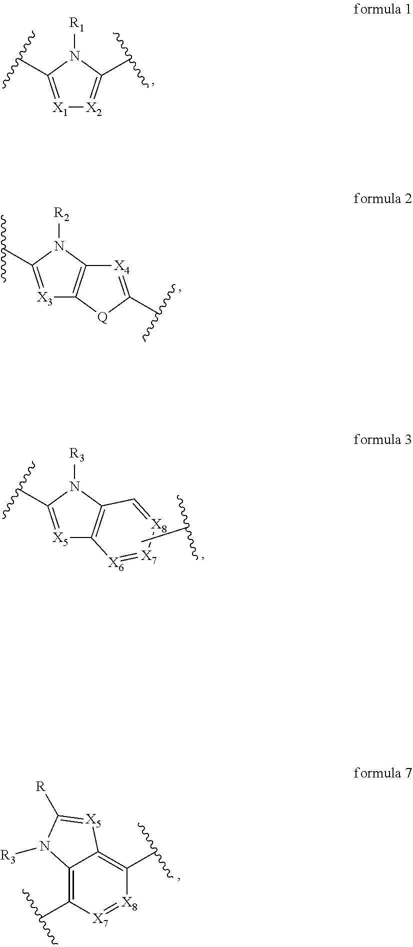
wherein the groups N are each independently selected from the following formulas: 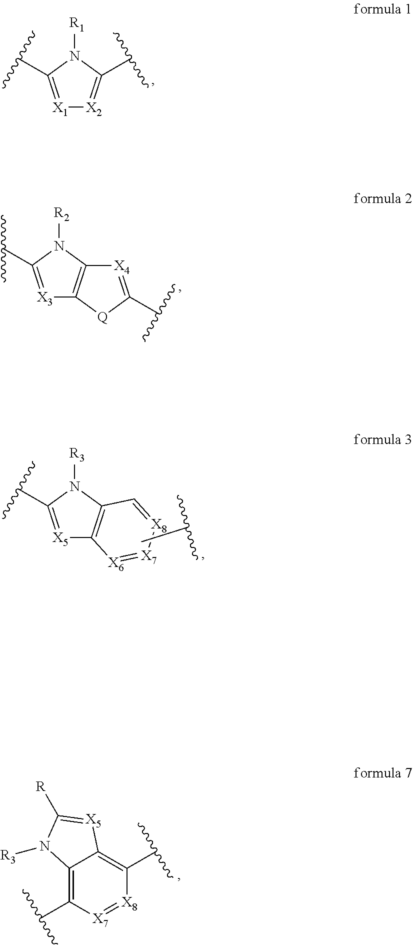
wherein M and N are each linked such that at least one N atom of the group M and an O atom of the group N are each bonded to one another via 2 C atoms, and [symbol] denotes the attachment to the groups in the compound of the general formula I, in which:
X₁-X₁₆ are independently selected from N or C—R, and in each of the groups of the formulas 3 and 6, a group from the groups X₈/X₇ and X₁₆/X₁₅ denotes the attachment [symbol] to other groups in the compound of the general formula I
R is in each case independently selected from the group consisting of H, halogen, branched or linear, cyclic or open-chain C₁-C₁₀ alkyl, C₂-C₁₀ alkenyl, O-alkyl, S-alkyl, O-alkenyl, S-alkenyl, alkynyl, aryl, and heteroaryl, wherein, in all of the groups from which R is selected, H atoms can be substituted, CN, NR₁₀R₁₁, with R₁₀ and R₁₁ each independently selected from H, branched or linear, and cyclic or open-chain C₁-C₁₀ alkyl, wherein H atoms of the C₁-C₁₀ alkyl can be substituted, and C atoms of the C₁-C₁₀ alkyl can be substituted by heteroatoms, and
R₁ to R₃ are each independently selected from the group consisting of H, branched or linear, and cyclic or open-chain C₁-C₁₀ alkyl, substituted or unsubstituted C₂-C₁₀ alkenyl, substituted or unsubstituted aryl, substituted or unsubstituted heteroaryl, and CN, wherein H atoms of the C₁-C₁₀ alkyl can be substituted, and C atoms of the C₁-C₁₀ alkyl can be substituted by heteroatoms,
wherein A1 and A2 are, independently, electron-withdrawing groups having at least one C═C double bond,
wherein the groups T1, T2, T3, and T4 are each independently selected from the following formulas: 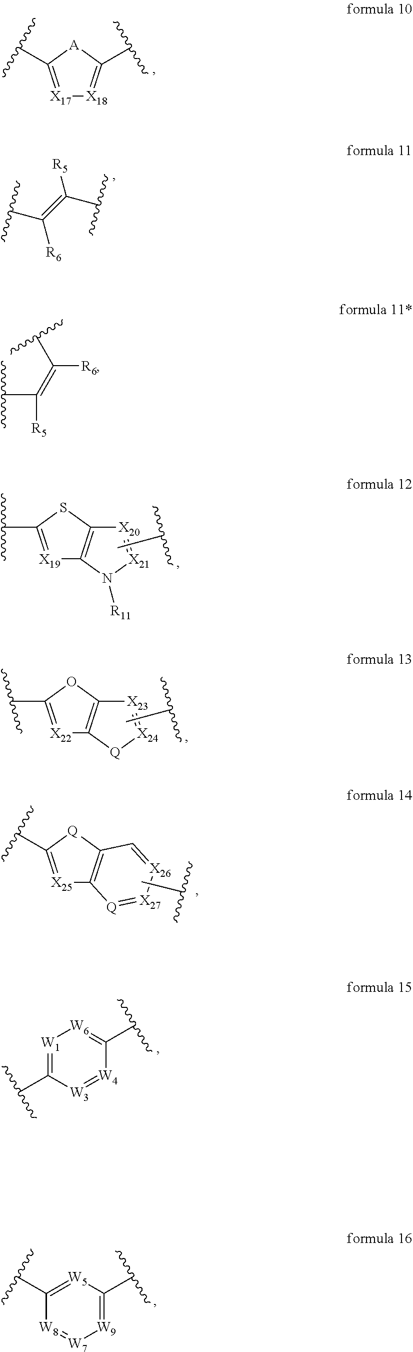
wherein [symbol] denotes the attachment to the other groups in the compound of the general formula I, and at least one of the groups T1 to T4 is the formula 10, in which:
R₅ and R₆ are each independently selected from the group consisting of H, CN, F, aryl, heteroaryl, C₂-C₁₀ alkenyl, alkynyl, branched or linear, cyclic or open-chain C₁-C₁₀ alkyl, wherein H atoms of the C₁-C₁₀ alkyl can be substituted, wherein, if the substituent R₁₃ is present in the compound of the formula I, a ring closure between R₅ with R₁₃ or R₆ with R₁₃ is possible, and, between R₅ and R₁₃ or between R₆ and R₁₃, in each case there is located the double bond from formula 11,
W₁ to W₈ are each independently selected from N, CR,
X₁₇ and X₁₈ are independently selected from N and C—R, and at least X₁₇ or X₁₈ is N, and
X₁₉ to X₂₇ are independently selected from N and C—R, and, in each of the groups of the formulas 12, 13, and 14, a group from the groups X₂₀/X₂₁, X₂₃/X₂₄, and X₂₆/X₂₇ denotes the attachment [symbol] to other groups in the compound of the general formula I, in which:
A is selected from the group consisting of S, O, NR₉, and Se, and
Q is selected from the group consisting of S, O, NR₉, and Se,
wherein, for the groups A and Q, the substituent R₉ is in each case independently selected from H, CN, branched or linear, cyclic or open-chain C₁-C₁₀ alkyl, C₂-C₁₀ alkenyl, O-alkyl, S-alkyl, O-alkenyl, S-alkenyl, alkynyl, substituted or unsubstituted aryl, and substituted or unsubstituted heteroaryl, wherein the H atoms of the C₁-C₁₀ alkyl may be substituted.

2: The compound according to claim 1, wherein the electron-withdrawing groups A1 and A2 are independently selected from the following formulas: 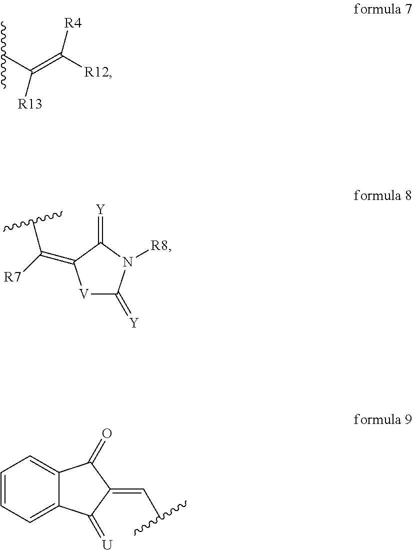
wherein [symbol] denotes the attachment to the groups T1 to T4 and Z in the compound of the general formula I, in which:
R₄ and R₁₂ are each independently selected from H, CN, and COOR, and R₄ and R₁₂ are not both H,
wherein R is selected from the same group of compounds as defined in R₁ to R₃,
wherein R₁₃ is in each case independently selected from the group consisting of H, CN, F, aryl, heteroaryl, C₂-C₁₀ alkenyl, alkynyl, branched or linear, cyclic or open-chain C₁-C₁₀ alkyl, wherein H atoms of the C₁-C₁₀ alkyl can be substituted,
wherein, if the substituent R₅ or R₆ is present in the compound of the formula I, a ring closure between R₅ with R₁₃ or R₆ with R₁₃ is possible, and, between R₅ and R₁₃ or between R₆ and R₁₃, in each case there is located the double bond from formula 11, in which:
V═O, S;
Y═O, S, C(CN)₂;
U═O, S, C(CN)₂, and
R₇ and R₈ are each independently selected from the group consisting of H, CN, F, aryl, heteroaryl, C2-C10 alkenyl, alkynyl, branched or linear, cyclic or open-chain C1-C10 alkyl, wherein H atoms of the C1-C10 alkyl may be substituted, and
wherein, for each of the groups A1 and A2, in each case for each C═C double bond in each case independently, both an E-isomer and a Z-isomer can be present.

3: The compound according to claim 1, wherein the electron-withdrawing groups A1 or A2 are the following groups: 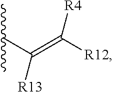
and
wherein R₄ and R₁₂ are CN.

4: The compound according to claim 1, wherein one of the parameters a, b, d, e equals 0, and/or
wherein at least one of the parameters a, b equals 1 and/or one of the parameters d, e equals 1.

5: The compound according to claim 1, wherein c=1, of the general formula II
A1-(T1)ₐ-(T2)_{b}-Z-(T3)_{d}-(T4)ₑ-A2 (II),
wherein Z is *-M-N—* or *—N-M-* and M is a group of

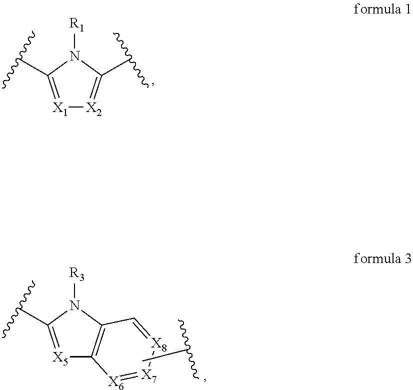

6: The compound according to claim 1, wherein c=1, having the general formula II
A1-(T1)a-(T2)b-Z-(T3)d-(T4)e-A2 (II)

7: The compound according to claim 1, wherein X₁₇ or X₁₈ is C—R, wherein R is independently selected from a group consisting of H, halogen, branched or linear, and cyclic or open-chain C₁-C₁₀ alkyl, R5 and R6 are each independently selected from H, CN, F, aryl, heteroaryl, C₂-C₁₀ alkenyl, alkynyl, branched or linear, and cyclic or open-chain C1-C10 alkyl, wherein H atoms of the C₁-C₁₀ alkyl may be substituted.

8: The compound according to claim 1, wherein X₁₇ and X₁₈ are N, and/or wherein X₁₉ to X₂₇ are independently selected from C—R,
wherein, in each of the groups of the formulas 12, 13, and 14, a group from the groups X₂₀/X₂₁, X₂₃/X₂₄, and X₂₆/X₂₇ denotes the attachment [symbol] to other groups in the compound of the general formula I.

9: The compound according to claim 1 wherein the group N is the following general group of formula 4

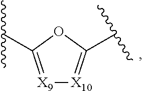

10: The compound according to claim 1, wherein M is the general group of formula 3 and/or N is the general group of formula 6.

11: The compound according to claim 1, wherein b=1 and T2 is the group of formula 10 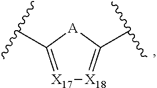
and/or wherein d=1 and T3 is the group of formula 10 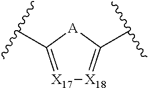
or formula 11

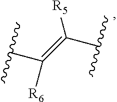

12: The compound according to claim 1, wherein e=1 and T4 is the group of formula 10 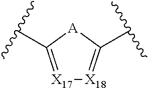
or formula 11 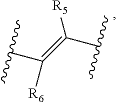
and/or wherein a=1 and T1 is the group of formula 10 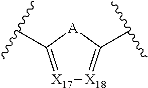
or formula 11

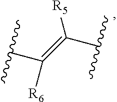

13: The compound according to claim 1, wherein the compound is selected from the group consisting of:

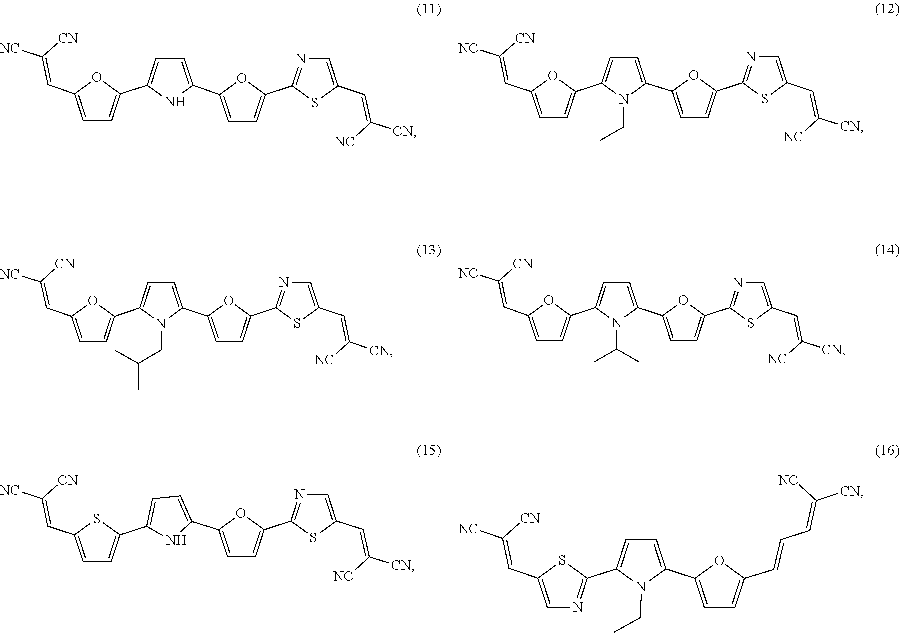 [SMILES: N#CC(C#N)=Cc1ccc(-c2ccc(-c3ccc(-c4ncc(C=C(C#N)C#N)s4)o3)[nH]2)o1 ; CCn1c(-c2ccc(C=C(C#N)C#N)o2)ccc1-c1ccc(-c2ncc(C=C(C#N)C#N)s2)o1 ; CC(C)Cn1c(-c2ccc(C=C(C#N)C#N)o2)ccc1-c1ccc(-c2ncc(C=C(C#N)C#N)s2)o1 ; CC(C)n1c(-c2ccc(C=C(C#N)C#N)o2)ccc1-c1ccc(-c2ncc(C=C(C#N)C#N)s2)o1 ; N#CC(C#N)=Cc1ccc(-c2ccc(-c3ccc(-c4ncc(C=C(C#N)C#N)s4)o3)[nH]2)s1 ; CCn1c(-c2ccc(/C=C/C=C(C#N)C#N)o2)ccc1-c1ncc(C=C(C#N)C#N)s1]

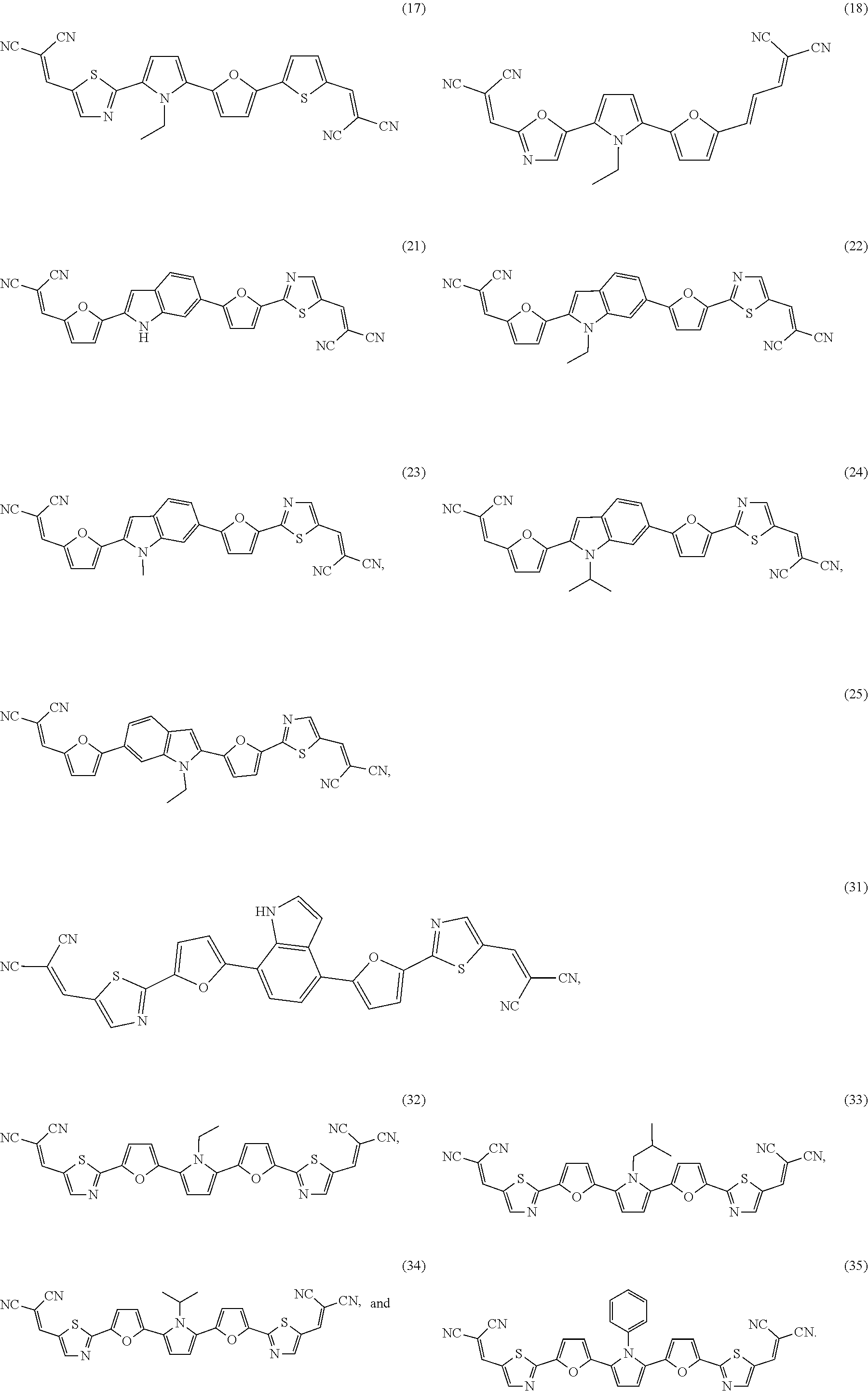

14: The compound according to claim 1, wherein c=1, b=1, and/or e=1.

15: An organic electronic device comprising the compound according to claim 1.

16: The organic electronic device according to claim 15, comprising:
an electrode;
a counter-electrode; and
at least one organic photoactive layer between the electrode and the counter-electrode,
wherein the organic photoactive layer comprises.
